(19) United States
(12) Patent Application Publication
(10) Pub. No.: US 2025/0290124 A1
(43) Pub. Date: Sep. 18, 2025

(54) ULTRA RAPID DROPLET DIGITAL POLYMERASE CHAIN REACTION

(71) Applicant: New York University, New York, NY (US)

(72) Inventors: Gilad EVRONY, New York, NY (US); Daniel ORRINGER, New York, NY (US); Zachary MURPHY, New York, NY (US)

(21) Appl. No.: 19/080,013

(22) Filed: Mar. 14, 2025

Related U.S. Application Data
(60) Provisional application No. 63/565,873, filed on Mar. 15, 2024.

(51) Int. Cl. C12Q 1/686; C12Q 1/48; C12Q 1/6853; C12Q 1/6886

(52) U.S. Cl. CPC C12Q 1/686; C12Q 1/485; C12Q 1/6853; C12Q 1/6886

(57) ABSTRACT

Compositions, devices and methods for performing an Ultra Rapid Droplet Digital Polymerase Chain Reaction (UR-ddPCR) are provided, for detecting and quantifying target nucleic acid obtained from a sample. The disclosed UR-ddPCR method includes an optional ultra-rapid DNA/RNA extraction method that is compatible with ddPCR and an ultra-rapid thermal cycling (UR-TC) method, the combination of which reduces the tissue-to-result time by about 83%, to ˜20 mins, preferably, <20 mins, when compared to ˜2 hrs required for standard ddPCR.

The thermal cycling time of UR-ddPCR is shortened compared to standard ddPCR to reduce the denaturation time from 30 seconds to about 1 to about 3 seconds and the annealing/extension step from 60 seconds to about 1 to about 10 seconds, eliminating final the 10-minute heat inactivation step used in standard commercial (Bio-Rad) ddPCR reactions.

### CROSS-REFERENCE TO RELATED APPLICATIONS

This application claims benefit of U.S. Provisional Application No. 63/565,873, filed Mar. 15, 2024, which is hereby incorporated herein by reference in its entirety. STATEMENT REGARDING FEDERALLY SPONSORED RESEARCH This invention was made with government support under grant number R01-CA226527 awarded by the National Institutes of Health. The government has certain rights in the invention.

### FIELD OF THE INVENTION

The disclosed invention is generally in the field of droplet digital PCR (ddPCR) and specifically in the area of ddPCR with increased speed.

### BACKGROUND OF THE INVENTION

Molecular-genetic information is critically important for managing cancer treatment, but it is not currently accessible in the operating room. The standard clinical workflow only provides this information several days or more than a week after surgery^{1,2}. Intraoperative knowledge of the molecular-genetic subtype of a tumor would be useful in guiding the surgical approach, and it would also avoid delays in planning post-surgical treatment. For example, stratification of a brain tumor as a low-grade versus high-grade glioma may justify a different resection strategy³. Additionally, the surgical decision of where to stop resecting tissue on the tumor margins is critical, but there is currently no technology to quantify the tumor cell percentage or density in a sample on the timescale required for intraoperative surgical guidance. In most surgeries, the boundaries of a tumor are determined by frozen histology and/or gross visualization by the surgeon, but this is imperfect, because it does not rely on molecular information. Ultra-rapid, intraoperative genetic subtyping and measurement of tumor cell percentage and density would therefore fundamentally change the approach surgeons take in resecting tumors and enable them to establish more optimal surgical endpoints.

Many cancer types frequently harbor one or more clonal mutations (i.e., mutations present in all the cells of the tumor) that are characteristic of that cancer type. These clonal hotspot mutations could allow for the development of widely applicable targeted intraoperative assays that both identify the molecular-genetic subtype of a tumor and quantify the tumor cell percentage of tissue samples to guide tumor resection. One example of such a hotspot mutation in glioma brain tumors is the IDH1 R132H mutation that is both frequent, occurring in approximately 65% of low-grade gliomas, and one of the most important prognostic biomarkers for adult gliomas^{4,5}. Another example is the BRAF V600E mutation that is frequently present in melanoma and also observed in many other tumor types, including thyroid, lung, ovarian, craniopharyngioma, and circumscribed low-grade gliomas^{6,7}. Each of these hotspot mutations is usually clonal^{8,9}, such that measuring the mutant DNA fraction of these mutations in a tumor sample quantifies the tumor cell percentage in that sample.

Notably, for gliomas and melanomas, as well as other tumor types, the completeness or extent of surgical resection is an important prognostic factor¹⁰⁻¹². Unfortunately, judging the boundaries of many tumors during surgery can be extraordinarily challenging¹³. Despite the introduction of a host of diagnostic modalities developed to help surgeons establish optimal endpoints for surgery, none can provide a rapid, direct, and accurate assessment of tumor cell burden at surgical margins¹². Additionally, for gliomas, the effects of surgery are more pronounced in IDH1-mutant than IDH1-wild type tumors¹⁴, so it would be greatly beneficial to tailor surgical objectives while taking IDH1 status into account. For these reasons, the IDH1 R132H and BRAF V600E mutations are prime candidates for an ultra-rapid intraoperative assay.

To be useful for clinical decisions in the operating room, any technology for molecularly subtyping a tumor and quantifying tumor cell percentage should be able to deliver results rapidly in <20 minutes with high sensitivity and specificity. Notably, there is no current technology meeting these requirements. Ultra-rapid histological methods can achieve the requirements for efficient integration into the surgical workflow¹⁵, but their results rely primarily on indirect inference based on tumor cell morphology rather than on genetic features that provide definitive tumor subtyping and detection. Previously, Shankar, et al. proposed a targeted quantitative PCR assay, but the tissue-to-result time was ˜60 minutes¹⁶. More recently, Wadden et al. developed a targeted sequencing method for rapid measurement of tumor cell percentage in a single sample in ˜30 minutes¹⁷, which represents a significant advance, but this is still not sufficiently fast for repeated intraoperative use.

Extreme PCR is a method for conducting PCR in <30 seconds¹⁸, but to date, it has only been implemented as a bulk assay that is unable to sensitively determine mutant DNA fraction in a sample, which would be necessary for measuring tumor cell percentage. Additionally, droplet-digital PCR (ddPCR), in which a bulk PCR reaction is partitioned into thousands of nanoliter-sized reactions, can measure tumor cell percentage with high sensitivity and specificity, but it currently takes >2 hours to perform, not including the time for extracting DNA from tissue.

A rapid method for detection and quantification of nucleic acids would also have applications beyond the operating room and surgeries, for example, for detection of infectious disease agents, especially in critical care settings such as emergency departments and intensive care units.

It is an object of the present invention to provide a method for droplet digital PCR with shortened tissue-to-result time useful in clinical and surgical settings.

It is also an object of the present invention to provide a method for rapidly detecting and quantifying a target nucleic acid in a sample.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### BRIEF SUMMARY OF THE INVENTION

Compositions, devices and methods for performing an Ultra Rapid Droplet Digital Polymerase Chain Reaction is provided, for detecting target nucleic acid obtained from a sample.

The disclosed UR-ddPCR method includes an optional ultra-rapid DNA/RNA extraction method (herein UR-Nucleic acid (NA) extraction) (herein, UR-NA extraction), that is compatible with ddPCR, and a method for ddPCR ultra-rapid thermal cycling (UR-TC). In some forms, the combination of these two processes reduces the tissue-to-result by about 83%, to ˜20 mins, preferably, <20 mins, when compared to ˜2 hrs required for standard ddPCR.

The UR-NA extraction method is used to extract DNA from a sample of interest. Nucleic acid molecules can be obtained directly from an organism or from a biological sample obtained from an organism, e.g., from blood, urine, cerebrospinal fluid, seminal fluid, saliva, sputum, stool and tissue. In some forms, the nucleic acid is DNA obtained from a tissue sample such as brain tissue.

When the sample is a tissue sample, the method includes at least three steps: (i) tissue homogenization for about 30 seconds in the presence of a nucleic acid extraction buffer that does not interfere with subsequent droplet formation, such as a detergent-free nucleic acid extraction buffer, (ii) heat incubation at about 98° C. for about 2.5 minutes, and (iii) a brief about 10-second centrifugation at a setting of ˜2,000×g to separate cellular debris from clarified lysate containing nucleic acid, to provide a complete processing time about 5 minutes for one tissue sample. UR-ddPCR may also utilize previously extracted nucleic acids which would not require the above UR-NA extraction. In some forms, the sample is not a tissue sample, and step (i) does not include a homogenization step, and it may not require step (iii). Alternative methods for rapid nucleic acid extraction, such as commercial kits (Lucigen QuickExtract and Qiagen QIAamp Fast DNA Tissue Kit) or microwave extraction may also be used when any utilized detergents are removed prior to the completion of the extraction process to avoid damage to subsequent ddPCR droplet formation.

UR-ddPCR thermal cycling (herein, UR-TC) has a cycling time that is shortened compared to standard ddPCR by eliminating the 10-minute enzyme activation step that is utilized in a standardddPCR reaction chemistry (for example, the commercial (Bio-Rad) ddPCR process), reducing the denaturation time from 30 seconds to about 1 to about 3 seconds, reducing the annealing/extension step from 60 seconds to about 1 to about 10 seconds, eliminating the 10-minute heat inactivation step, placing the reaction mix containing droplets in a container with high thermal conductance and effective surface area to volume ratio, for example, a stainless-steel capillary, and conducting thermal cycling using heating elements that are pre-heated to each of the required temperature steps of PCR. The pre-heated heating elements increase the speed of thermal cycling by avoiding the repeated ramping between the PCR temperature steps that a standard thermal cycler performs in standard ddPCR. In some forms, the heating element used for thermal cycling is a hot water bath.

The UR-ddPCR method includes the steps of: (a) contacting a sample containing target nucleic acid (either previously extracted, or extracted by the UR-NA extraction method) with an effective amount of a UR-ddPCR reaction mix which includes components necessary for a PCR reaction; (b) generating water-in-oil emulsion droplets wherein the water phase contains the target nucleic acids and the UR-ddPCR reaction mix; and (c) UR-TC. The UR-ddPCR reaction mix contains: (a) a DNA polymerase, preferably, an aptamer-inhibited hot-start polymerase that does not require a heat activation step required by some hot-start polymerases; (b) optionally, a suitable restriction enzyme to reduce the average size of DNA fragments so they partition more evenly in droplets or a glycosylase enzyme to remove DNA damage or dUTPs incorporated by prior reactions to avoid cross-reaction PCR contamination; (c) dNTPs; (d) magnesium salt required for DNA polymerase activity; (e) other salts and a buffer to stabilize pH that are standard in PCR; (f) primers specific for the target nucleic acid; and (g) a probe labeled with a fluorophore and a fluorescence quencher (i.e., a TaqMan type probe). Probes preferably include locked nucleic acids. Multiple target nucleic acid sequences may be detected in a multiplexed reaction incorporating additional primers and probes.

In step (a) of UR-ddPCR, a DNA sample is contacted with a polymerase, primers, and probes in amounts effective to allow for amplification with denaturation in as little as 1 to 3 seconds and annealing/extension in as little as 1 to 10 seconds. The polymerase is provided at a final reaction concentration of at least 0.15 U/μl, (where 1U (unit) is the amount is the amount of enzyme that incorporates 15 nmol of dNTP into acid insoluble material in 30 minutes at 75°° C.), preferably, at a final reaction concentration from about 0.15 U/μL to about 1 U/μL (6× to 40×, where 1× concentration is 0.025 U/μL); primers are each provided at a final reaction concentration of at least 1.8 μM to 10.8 μM each (2× to 12×, where 1× concentration is 0.9 μM each); and probes at a final reaction concentration from about 0.375 μM to about 3 μM (1.5× to 12×, where 1× concentration is 0.25 μM each).

In step (b) of UR-ddPCR, droplets including the target DNA are generated. The components of step (a) are mixed with droplet generation oil and used to create a water-in-oil emulsion that partitions the PCR reaction into thousands of droplets. The droplets are spherical with volumes ranging from about 0.1 to 10 nL.

Step (c) of UR-ddPCR is the UR-TC step, and it includes cycling the UR-ddPCR droplets between at least a denaturation temperature and an annealing/extension temperature through a plurality of amplification cycles where each cycle is completed in about 2 to less than about 13 seconds, per cycle, for example, in about 2, 3, 4, 5, 6 7, 8, 9, 10, 11, 12, or 13 seconds per cycle, wherein the droplets are loaded into a droplet container with high-thermal conductance such as thin-walled stainless steel capillaries, wherein the plurality of cycles is at least 30, 35, 40, 45 or 50 cycles. For example, denaturation can be completed between 1 to about 3 seconds, and annealing/extension can be completed between 1 to about 10 seconds. Thermal cycling may also include about 1 second to transfer the sample between each temperature step. Optionally, thermal cycling may be performed by cycling the sample between three temperature steps: denaturation, annealing, and extension (i.e., annealing and extension are separated into two separate steps at different temperatures).

After UR-TC, fluorescence signals of droplets can be quantified by either a standard droplet reader (for example, Bio-Rad QX200 instrument) or other fluorometric detection methods such as a flow cytometer, a fluorescence microscope, or fluorescence excitation source and photomultiplier tube detector.

Also provided is a method for detecting and quantifying a target nucleic acid in a sample using UR-ddPCR as disclosed herein.

In some forms, the methods are used to detect and quantify a mutation in a target DNA, where the target nucleic acid includes a mutation. The method includes performing UR-ddPCR on a sample. In some forms, the sample is a tissue sample obtained from a subject.

In some forms, the method detects and quantifies an IDH1 R132H mutation in a sample obtained from a subject. In some forms, the method detects and quantifies a BRAF V600E mutation in a sample obtained from the subject.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIG. 1A is a schematic of Tissue-to-result Ultra-Rapid ddPCR. FIG. 1B shows microscopy images of ddPCR droplets after droplet generation from a reaction mix containing an additional 4 μL of each specified DNA extraction buffer. Scale bars, 100 μm. FIG. 1C is a schematic of Ultra-Rapid (UR) DNA extraction. s, seconds. FIG. 1D shows the total time of UR-DNA extraction when processing 1 sample at a time (N=3 replicates) and 4 samples in parallel (N=3 replicates). FIG. 1E is a schematic of how ddPCR signal plots shown in the remaining figures correspond to raw ddPCR 2-dimensional (2D) plots. ddPCR signal plots show the mean fluorescence signal amplitude (a.u., arbitrary units) of the double-negative population and each single-positive population with error bars of +2 standard deviations (std). Note that the signal level of the double-positive population (i.e., mutant+/non-mutant+) is not depicted in ddPCR signal plots. Additionally, we do not depict positive populations on ddPCR signal plots if they contain 3 or fewer droplets, as these cannot be distinguished from rare false positive events. FIG. 1F is a ddPCR signal plot of a standard ddPCR assay for IDH1 R132H DNA with input from either a standard DNA extraction or UR-DNA extraction. The total number of ddPCR droplets measured for each assay are listed below each sample.

FIG. 2A is a ddPCR signal plot for the IDH1 R132H assay with multiple thermal cycling and ddPCR reaction mix conditions that reduce the total thermal cycling time (m, minutes; s, seconds). All modified parameters are listed for each sample, and the parameters that were constant for all samples are listed in the “Constant Parameters Across Conditions” section. 1× primer and 1× probe concentrations refer to the concentrations used in standard ddPCR (0.9 μM each primer and 0.25 μM each probe), and the 1× Aptamer Hot-Start (HS) Taq concentration refers to 0.025 U/μL. The modified and constant parameters for all the other ddPCR signal plots in this figure are described in the same manner. a.u., arbitrary units.

FIG. 2B is a schematic comparison of the properties of a standard ddPCR 96-well plate's well and a stainless-steel capillary with a depiction of the capillary thermal cycling process. See the Methods for calculation details. FIG. 2C is a ddPCR signal plot for the IDH1 R132H assay showing the effect of switching to the stainless-steel capillary and water bath thermal cycling system. FIG. 2D is a ddPCR signal plot for the IDH1 R132H assay showing the effect of increasing the Aptamer HS Taq concentration, in the presence of higher 4× primer and probe concentrations. FIG. 2E is a schematic of the estimated standard ddPCR and UR-ddPCR thermal cycling times. The standard ddPCR bar depicts the times of each standard ddPCR step. The thermocycler ramp time was calculated as the difference between the total PCR run time and the sum of the times of individual thermal cycling steps. The UR-ddPCR bar depicts the time of the denaturation and annealing/extension steps (1 s per cycle) and typical time required for manually transferring the capillary between water baths (1 s per water bath transfer; 2 transfers per cycle).

FIG. 3A is a representative two-dimensional ddPCR plot from an IDH1 R132H UR-ddPCR assay of 50% IDH1 R132H mutant reference DNA. The method for marking negative and positive droplet populations is described in the Methods section. FIG. 3B shows sensitivity of the IDH1 R132H assay by UR-ddPCR compared to standard ddPCR with DNA inputs containing known percentages of IDH1 R132H mutant DNA. The red line (y=x) depicts theoretical identical measurements by standard ddPCR and UR-ddPCR. This experiment used 32X Aptamer HS Taq concentration. See Figure S1E for data of an experiment using 25X Aptamer HS Taq concentration. FIG. 3C is a representative two-dimensional ddPCR plot from a BRAF V600E UR-ddPCR assay of 50% BRAF V600E mutant reference DNA. The method for marking negative and positive droplet populations is described in the Methods section. FIG. 3D is a sensitivity of the BRAF V600E assay by UR-ddPCR compared to standard ddPCR with DNA inputs that containing known percentages of BRAF V600E mutant DNA. This experiment used 25X Aptamer HS Taq concentration. The red line depicts the theoretical exact measurement of the input mutant DNA percentage.

FIG. 4A is a representative two-dimensional ddPCR plot of a tissue-to-result UR-ddPCR assay of an IDH1 R132H mutant tumor tissue sample. a.u., arbitrary units. FIG. 4B shows mutant DNA percentages of IDH1 R132H mutant tumor samples measured by tissue-to-result UR-ddPCR compared to standard ddPCR of the same UR-DNA extraction extracts. In each experiment, tumor samples were profiled either one at a time or four samples in parallel. Error bars, 95% Poisson confidence intervals. Significant differences: p<0.05 (*), as measured by the Poisson difference of means test. FIG. 4C shows the step-by-step time of the tissue-to-result UR-ddPCR assays shown in FIG. 4B.

FIG. 5A is a schematic of intraoperative tissue-to-result UR-ddPCR. FIG. 5B shows intraoperative tumor cell percentages measured by tissue-to-result UR-ddPCR compared to measurements of the same samples by standard ddPCR. We colored separately samples profiled with the intraoperative UR-ddPCR protocol version 1 (v1) versus version 2 (v2) of the IDH1 R132H assay, which differ in how the UR-ddPCR reaction mix was prepared, and also colored separately samples profiled with the BRAF V600E assay (all of which were profiled with protocol version 2). Error bars, 95% Poisson confidence intervals. See FIGS. 9A and 9B for tumor cell percentage and estimated tumor cellularity plots of each sample grouped by surgical case. FIG. 5C shows the step-by-step time of all intraoperative tissue-to-result UR-ddPCR cases.

FIGS. 6A-6E show additional Ultra-Rapid ddPCR thermal cycling optimizations, related to FIG. 2. FIG. 6A shows ddPCR signal plot for the IDH1 R132H assay showing the effect of decreasing the annealing/extension time from 15 seconds to 1 second. FIG. 6B shows ddPCR signal plot of increasing numbers of PCR cycles. For this and subsequent figure panels, all modified parameters are listed under each sample and parameters that were constant for all samples are listed in the “Constant Parameters Across Conditions” section. a.u., arbitrary units; m, minutes; s, seconds. FIG. 6C shows ddPCR signal plot of increasing primer concentration. FIG. 6D shows ddPCR signal plot of increasing probe concentration. FIG. 6E shows sensitivity of the IDH1 R132H assay by UR-ddPCR compared to standard ddPCR with DNA inputs containing known percentages of IDH1 R132H mutant DNA. The red line (y=x) depicts theoretical identical measurements by standard ddPCR and URddPCR. This experiment used 25X Aptamer HS Taq concentration. See FIG. 3B for data of an experiment using 32X Aptamer HS Taq concentration.

FIG. 7 shows the effect of probe chemistry on UR-ddPCR performance, related to FIG. 2. Comparison of UR-ddPCR fluorescence signal amplitudes when using PrimeTime locked nucleic acid versus Affinity Plus probe chemistries with a HEX fluorophore, for a BRAF (nonmutant sequence) probe assaying control DNA with the same protocol for each probe type. X-axis, droplets in order of instrument reading. a.u., arbitrary units.

FIGS. 8A-8C show layouts of intraoperative tissue-to-result Ultra-Rapid ddPCR mobile carts, related to FIG. 5. FIG. 8A shows tissue processing table. FIG. 8B shows pre-PCR and droplet reader table. Note, in the ‘Top Shelf’, the 200 μL Pipette Tips and 200 μL Pipette were from the brand Rainin for cases #1-3 as shown in the photograph, but subsequently changed to 200 μL Corning brand pipette tips and a 200 μL Eppendorf Pipette. FIG. 8A shows the post-PCR table.

FIGS. 9A-9E show intraoperative UR-ddPCR measurements of control DNA mutant DNA percentages and negative case tumor cell percentages, related to FIG. 5. FIGS. 9A and 9B show mutant DNA percentages measured with the IDH1 R132H assay for negative control DNA (FIG. 9A) and for 50% mutant IDH1 R132H control DNA (FIG. 9B) (one control sample per case assayed by both UR-ddPCR and standard ddPCR, except cases 5 and 6 that shared the same control sample as they were performed on the same day). Horizontal lines, average of values. FIG. 9C shows tumor cell percentages of all tissues samples from IDH1 R132H-negative cases measured by intraoperative UR-ddPCR and by standard ddPCR of the same DNA extract. Cases are classified as IDH1 R132H-negative based on clinical sequencing. Horizontal lines, average of values. FIGS. 9D and 9E show mutant DNA percentages measured with the BRAF V600E assay for negative control DNA (FIG. 9D) and for 50% mutant BRAF V600E control DNA (FIG. 9E) (one control sample per case assayed by both UR-ddPCR and standard ddPCR, except cases 5 and 6 that shared the same control sample as they were performed on the same day). Horizontal lines, average of values.

FIGS. 10A and 10B show intraoperative UR-ddPCR measurements of tumor cell percentages and cellularities, grouped by surgical case, related to FIG. 5.

FIG. 10A shows tumor cell percentages measured by intraoperative tissue-to-result UR-ddPCR compared to standard ddPCR of the same samples. Error bars, 95% Poisson confidence intervals (not shown when less than size of plotted point). FIG. 10B shows estimated tumor cellularities calculated as [NIO total cellularity]×[tissue-to-result UR-ddPCR tumor cell percentage]. Cases are classified as mutant or non-mutant based on clinical sequencing. Error bars, 95% Poisson confidence intervals (not shown when less than size of plotted point).

### DETAILED DESCRIPTION OF THE INVENTION

The disclosed method and compositions can be understood more readily by reference to the following detailed description of particular embodiments and the Example included therein and to the Figures and their previous and following description.

The disclosed compositions and methods demonstrate an ultra-rapid (UR)-ddPCR method that achieves a total tissue-to-result time of 15 minutes. The disclosed method achieves comparable sensitivity for quantifying target nucleic acids as standard ddPCR. A compatible ultra-rapid DNA extraction procedure is disclosed herein. UR-ddPCR assays are demonstrated for both the IDH1 R132H and G12CBRAF V600E mutations, the application of the disclosed methods in an operating room demonstrated by measuring tumor percentage in the operating room for glioma surgeries-the first demonstration of an ultra-rapid (<20 minutes) quantitative genetic assay in the operating room.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### I. DEFINITIONS

The term “conditions sufficient for” refers to any environment that permits the desired activity, for example, that permits specific binding or hybridization between two nucleic acid molecules or that permits reverse transcription and/or amplification of a nucleic acid. Such an environment may include, but is not limited to, particular incubation conditions (such as time and/or temperature) or presence and/or concentration of particular factors, for example in a solution (such as buffer(s), salt(s), metal ion(s), detergent(s), nucleotide(s), enzyme(s), etc.).

As used herein, the terms “nucleic acid”, “polynucleotide” and “oligonucleotide” refer to primers, probes, oligomer fragments, and oligomer controls and are generic to polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double-and single-stranded DNA, as well as double-and single stranded RNA.

As used herein, the terms “detect” or “detecting” generally refer to obtaining information. Detecting or determining can utilize any of a variety of techniques available to those skilled in the art, including for example specific techniques explicitly referred to herein.

Detecting may involve manipulation of a physical sample, consideration and/or manipulation of data or information, for example utilizing a computer or other processing unit adapted to perform a relevant analysis, and/or receiving relevant information and/or materials from a source. Detecting may also mean quantifying the level of a target analyte, for example, the fraction of input nucleic acids from a target locus that have a specific mutation. Detecting may also mean comparing an obtained value to a known value, such as a known test value, a known control value, or a threshold value. Detecting may also mean forming a conclusion based on the difference between the obtained value and the known value.

The terms “contact”, “contacting” or “bringing into contact” describe placement in physical association for example, in solid and/or liquid form. For example, contacting or combining can occur in vitro with one or more primers and/or probes and a biological sample (such as a sample including nucleic acids) in solution.

“Amplification” or “amplifying” refers to increasing the number of copies of a nucleic acid molecule, such as a gene, fragment of a gene, or other genomic region. The products of an amplification reaction are called “amplification products” or “amplicons.”

As used herein, the term “primer” refers to an oligonucleotide, which is capable of acting as a point of initiation of nucleic acid synthesis when placed under conditions in which synthesis of a primer extension product which is complementary to a target nucleic acid strand is induced, e.g., in the presence of different nucleotide triphosphates and a polymerase in an appropriate buffer (“buffer” includes pH, ionic strength, cofactors etc.) and at a suitable temperature. In some embodiments, the primer is preferably single-stranded. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the template. Primer includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of a double-stranded DNA (dsDNA) fragment. A “reverse primer” anneals to the sense-strand of a dsDNA fragment. The terms “primer pair” and “primer set” refers to a forward and reverse primer pair (i.e., a left and right primer pair) that can be used together to amplify a given region of a nucleic acid of interest.

The terms “hybridization probe” or “probe” as used interchangeably herein are oligonucleotides capable of binding in a base-specific manner to a complementary strand of nucleic acid. Such probes include nucleic acids, peptide nucleic acids, as described in Nielsen et al., Science 254, 1497-1500 (1991), and other nucleic acid analogs and nucleic acid mimetics. Probes may also include TaqMan type probes that contain both a fluorophore and a fluorescence quencher which are separated from each other to generate a fluorescence signal when they hybridize to a target nucleic acid sequence followed by digestion by a DNA polymerase with 5′->3′ nuclease activity.

The term “label” as used herein refers to a moiety that directly or indirectly facilitates detection of a molecule by providing a detectable signal. Common labels include fluorescent, luminescent, light-scattering, and/or colorimetric labels.

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in naturally occurring nucleic acids may be included in the nucleic acids described herein. These include, for example, inosine, 7-deazaguanine, Locked Nucleic Acids (LNA), and Peptide Nucleic Acids (PNA). Complementarity need not be perfect (e.g., it can be partial or complete); stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

The terms “target nucleic acid” or “target sequence” or “target segment” as used herein refer to a nucleic acid sequence of interest to be detected and/or quantified in the sample to be analyzed. Target nucleic acid may be composed of segments of a genome, a complete gene with or without intergenic sequence, segments or portions of a gene with or without intergenic sequence, or sequence of nucleic acids to which probes or primers are designed to hybridize. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

“Subject” as used herein refers to either a human or non-human animal. As used herein, the term “sample” refers to in vitro samples as well as clinical samples obtained from a patient. In preferred embodiments, a sample is obtained from a biological source (i.e., a “biological sample”), such as tissue or bodily fluid collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy or surgically resected material), nasopharyngeal aspirate, and other discussed herein and otherwise known in the art.

“Unit” or “U” when used in context of an enzyme refers to an amount of enzyme required to convert a given amount of reactant or substrate to a product using a defined time and temperature.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. COMPONENTS

UR-ddPCR includes various components disclosed herein, the combination of which allows for tissue-to-result time of <20 mins. In some forms, the sample is not a tissue sample. For example, the sample could be a liquid sample, or a pre-extracted DNA sample. Components include, but are not limited to a UR-ddPCR mix which includes the necessary components required for dd-PCR, like primers and probes/hybridization probes for amplifying and detecting nucleic acids of interest (target nucleic acid), DNA polymerase, dNTPs, other optional enzymes; a means for generating droplets; a droplet container preferably made from a material with a high thermal conductance and surface area to volume ratio; a means for rapid thermal cycling which includes a pre-heated heating element such as water baths, and a means for reading droplet fluorescence.

### A. Primers and Probes

The disclosed methods include the use of primers and/or probes that are specific to target nucleic acid, such as a specific mutation of interest. The primers and probes are used for amplification and/or detection to detect and determine the identity of one or more nucleic acids of interest. UR-ddPCR primer targeting a mutation of interest are designed with the following considerations. The primers are designed to have short amplicons, preferably, <200 base pairs, for example between 40 and 200 base pairs, for example, 50, 60, 70, 80, 90, 100, 110, 120, 130, 140, 150, 160, 170, 180, 190 base pairs to reduce the time necessary for the polymerase to replicate the amplicon-thereby lowering the required annealing/extension time in PCR.

The disclosed methods use probes, which are small nucleic acid sequences labeled with a reporter molecule. The labeled sequence, or probe, is very specific and recognizes complementary sequences of the target DNA. The probes may be no more than 10, 15, 18, 20, 25, 30, 35, 40, 45, 50, 55, or 60 nucleotides in length. Preferably, probes are designed with locked nucleic acids to enable shorter probes, which in turn increases the difference in melting temperatures for a matched versus mismatched base at the mutation location. A locked nucleic acid (LNA) is a modified RNA nucleotide in which the ribose moiety is modified with an extra bridge connecting the 2′ oxygen and 4′ carbon. The bridge “locks” the ribose in the 3′-endo (North) conformation, which is often found in the A-form duplexes.

Amplification of the target can be detected based on the detectably labeled probe. In some forms, amplification of the target can be detected by detecting a probe labeled with a fluorophore and a fluorescence quencher included in the reaction (i.e., Taqman type probe).

In some forms, the detectably labeled probes are optically labeled probes, such as fluorescently labeled probes. Examples of fluorescent labels include, but are not limited to,

Atto dyes, 4-acetamido-4′-isothiocyanatostilbene-2,2′ disulfonic acid; acridine and derivatives: acridine, acridine isothiocyanate; 5-(2′-aminoethyl) aminonaphthalene-1-sulfonic acid (EDANS); 4-amino-N-[3-vinylsulfonyl) phenyl] naphthalimide-3,5 disulfonate; N-(4-anilino-1-naphthyl) maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino] naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl) aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123,rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are FAM and VICTM (from Applied Biosystems). Labels other than fluorescent labels are contemplated by the invention, including other optically-detectable labels.

In some forms, the probe is labeled with a fluor/quencher pair or a FRET pair. In both cases of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair,” both of which terms are discussed in more detail below. The term “fluorescence-emitting dye pair” is used interchangeably with the phrase “a FRET pair and/or a quencher/fluor pair.” A quencher/fluor pair when used with a probe, includes a fluorescent reporter dye on one end of the probe and a quenching element on the other side that prevents fluorescence by absorbing the light emitted by the reporter. The fluorescent reporter dye and the quencher are located close to each other in order for the quencher to prevent fluorescence. Hydrolysis probes, called TaqMan probes, are sequence-specific oligonucleotides with a bound fluorophore and quencher moiety. A fluorophore binds to the 5′ end of the probe, and a quencher binds to its 3′ end or internally within the sequence of interest. During the extension phase of PCR, the probe is cleaved by the 5′->3′ exonuclease activity of Taq DNA polymerase, separating the fluorophore and the quencher moiety. As a result, a fluorescence signal proportional to the amount of accumulated PCR product can be detected.

Any convenient donor acceptor pair (signal moiety/quencher moiety pair) can be used and many suitable pairs are known in the art. In some cases, a quencher moiety is a dark quencher. A dark quencher can absorb excitation energy and dissipate the energy in a different way (e.g., as heat). Thus, a dark quencher has minimal to no fluorescence of its own (does not emit fluorescence). Examples of dark quenchers are further described in U.S. Pat. Nos. 8,822,673 and 8,586,718; U.S. patent publications 20140378330, 20140349295, and 20140194611; and international patent applications: WO200142505 and WO200186001, all if which are hereby incorporated by reference in their entirety. In some cases, a detectable label is a fluorescent label selected from: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, and Pacific Orange. Examples of AlexaFluor dyes include, but are not limited to: Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, Alexa Fluor® 790, and the like. Examples of quencher moieties include, but are not limited to: a dark quencher, a Black Hole Quencher® (BHQ®) (e.g., BHQ-0, BHQ-1, BHQ-2, BHQ-3), a Qx1 quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

### B. UR-ddPCR Mix

The UR-ddPCR (Droplet Digital PCR) mix includes the necessary components (in addition to the primers and probes designed for the target sequence, and the sample DNA template) required for ddPCR, like DNA polymerase (a heat-stable enzyme responsible for amplifying the target DNA sequence); dNTPs (deoxynucleotides of type A, C, G and T, which are building blocks for the new DNA strand during amplification); water, salts and pH-stabilizing buffers required for DNA polymerase activity; optional additives such as DMSO to aid in denaturing DNA; and other optional enzymes such as restriction enzymes for reducing average DNA fragment size or uracil DNA glycosylase to remove previously amplified fragments if these were amplified with dUTPs or a reverse transcriptase to convert DNA to RNA. This reaction mix is input into a droplet generator that creates a water-in-oil emulsion, with the reaction mix comprising the water phase of the emulsion, that partitions the PCR reaction into thousands of droplets.

The ddPCR Supermix for Probes, no dUTP (Bio-Rad) may also be used as an example starting formulation for the UR-ddPCR mix, to which are then added primers and probes in a final reaction concentration that is 4× the concentrations used in standard ddPCR (where 1× concentrations are 0.9 μM each for primers and 0.25 μM each for probes) a rapid hot-start polymerases such as Aptamer HS Taq polymerase in a final reaction concentration of about 25× or about 32X relative to the 0.025 U/μL concentration recommended by NEB (New England Biolabs) for standard PCR.

Aptamer HS Taq polymerase is a DNA polymerase that uses a DNA aptamer to control when the polymerase is active. This allows for PCR reactions to be set up at room temperature. The aptamer binds to the polymerase, blocking its activity at room temperature, which prevents non-specific amplification from occurring at low temperatures. The aptamer rapidly dissociates from the polymerase at higher temperatures. The rapid activation of aptamer-based hot start polymerases is useful for UR-ddPCR, as these avoid the need for a prolonged initial heat activation step at the beginning of the thermal cycling.

In general, the polymerase is provided at a final reaction concentration of at least 0.15 U/μl, (where 1U (unit) is the amount is the amount of enzyme that incorporates 15 nmol of dNTP into acid insoluble material in 30 minutes at 75° C.), preferably, at a final reaction concentration from about 0.15 U/μL to about 1 U/μL (6× to 40×, where 1× concentration is 0.025 U/μL); primers are each provided at a final reaction concentration of at least 1.8 μM to 10.8 μM each (2× to 12×, where 1× concentration is 0.9 μM for each primer); and probes at a final reaction concentration from about 0.375 μM to about 3 μM (1.5× to 12×, where 1× concentrations is 0.25 μM for each probe).

### C. Droplet Container for Thermal Cycling

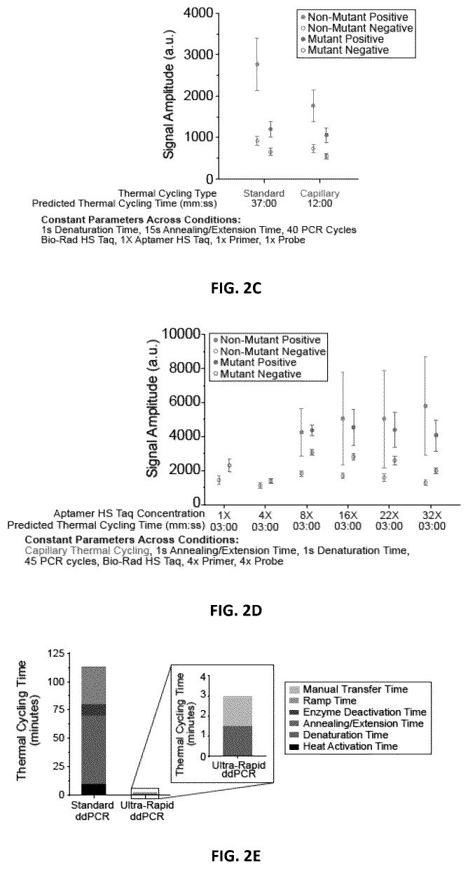

The thermal cycling system includes a droplet container with an effective thermal conductance and surface area to volume ratio required to obtain the dramatic reduction in the thermal cycling time when compared to standard ddPCR (FIGS. 2B and 2E) i.e., the container is effective for UR-TC, in that a detectable fluorescence signal is generated when each UR-TC PCR cycle's denaturation step lasts from about 1 to about 3 seconds and the sum of each UR-TC PCR cycle's annealing and extension steps (or each PCR cycle's annealing/extension step when annealing and extension are combined in one step) lasts from about 1 second to about 10 seconds. An effective thermal conductance, which depends both on the material and its thickness is at least 5,000 W/(m²*K), and an effective surface area to volume ratio is at least 1.5 mm²/μL and up to 6 mm²/μL, for example, 2, 2.5, 3, 3.5, 4, 4.5, 5, 5.5 mm²/μ. Suitable materials for the droplet container include, but are not limited to metal (steel, aluminum, copper, silver), glass, polypropylene, polycarbonate, silicon carbide, in various shapes such as tubing, standard plate wells, capillaries, or microfluidic channels. In some forms, the droplet container is a stainless steel capillary tube, as disclosed herein.

Rather than load droplets generated during UR-ddPCR into a 96-well plate, the droplets are loaded into the droplet container that has an effective thermal conductance and surface area to volume ratio for rapid cycling, such as thin-walled stainless steel capillaries.,

In some forms the droplets container is a stainless steel capillary (i.e., a hollow cylinder) preferably with the following properties: 304 stainless steel, 18 gauge extra thin wall hypodermic tubing×2.25 inches long, 0.0495 to 0.0505 inches outer diameter, 0.041 to 0.043 inches inner diameter, welded and drawn, burr-free. Preferably, the capillaries are subjected to a standard wash followed by an ethanol wash. The thermal conductivity of stainless-steel 304, the material of capillaries used herein, is approximately 15 W/(m*K)³⁷.

The thickness of the stainless-steel capillary is 0.10 +0.04 mm; and the thermal conductance for the stainless-steel capillary is approximately 1.5×10⁵ W/(m²*K) (which is an ˜204-fold difference from that of the standard ddPCR plate well. The surface area to volume ratio of the capillary is 3.75 mm²/μL, which is 3.8-fold higher than that of the ddPCR plate well, as calculated herein.

### D. Heating Elements

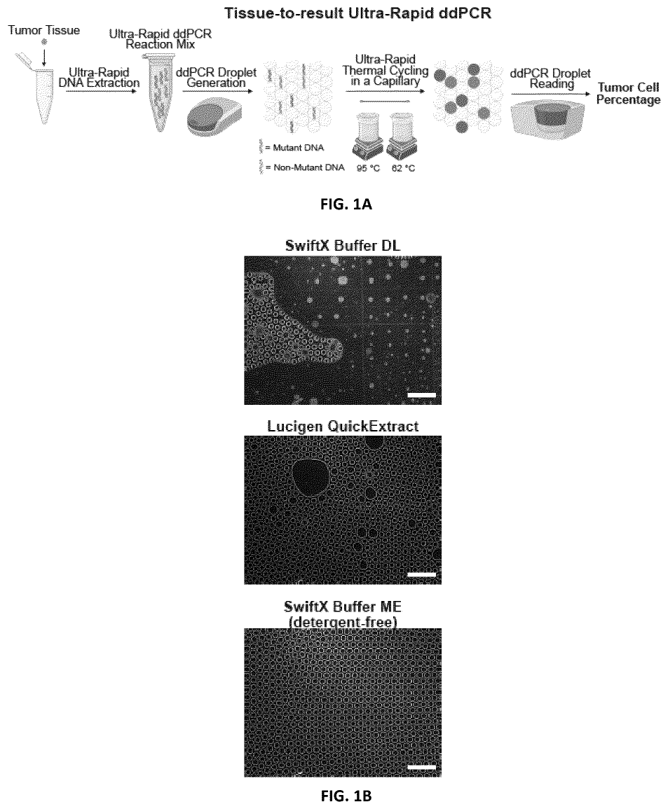

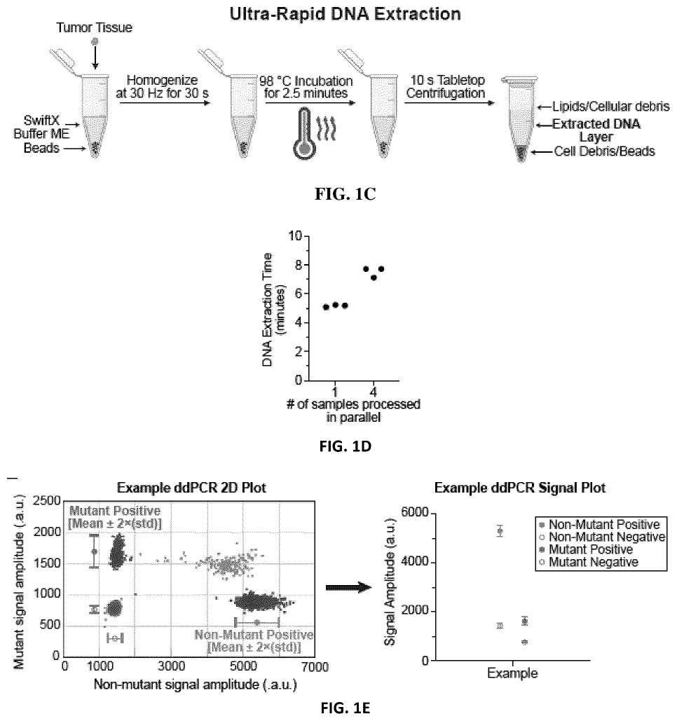

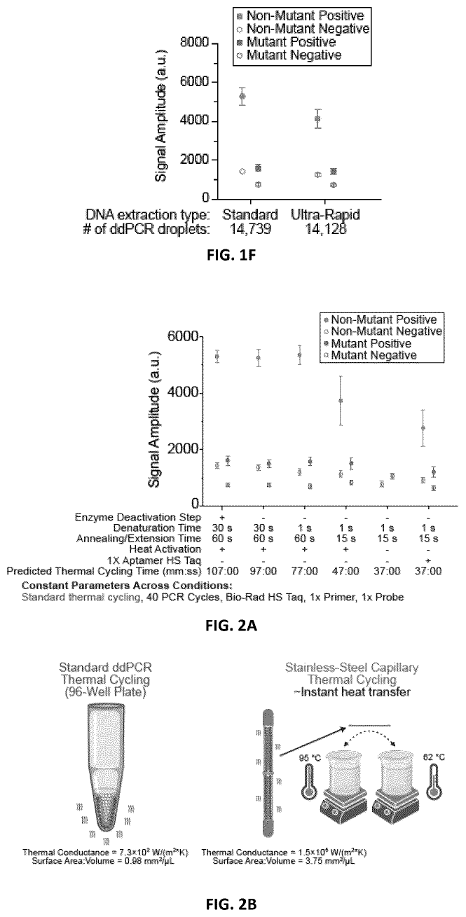

Thermal cycling includes contacting the droplet container with heating elements. The heating elements are preferably pre-heated to the disclosed thermal cycling temperatures. Examples of heating elements include, but are not limited to, heated water (water baths), thermoelectric heating elements (for example, Peltier elements), resistive heating elements (for example metal strip, coil, or ribbon elements made of nichrome, Kanthal, or stainless steel), radiating heating elements such as infrared (for example, ceramic heated bodies) or microwave, heated oil (for example, Sigma Aldrich Silicone Oil AR 20 or Paratherm NF Heat Transfer Fluid), and heated fluorocarbon-based liquids (for example, 3M Fluorinert FC-3283 or FC-40). In some forms where the heating element is heated water, prior to performing UR-ddPCR, the following can be prepared: Two 1-liter water baths, one at 95° C. and one at 62° C. The water baths can be heated on hot plates with continuously active magnetic stir bars, and temperatures continuously measured with standard thermometers. FIG. 1A.

### E. Nucleic Acid Target Molecules

Nucleic acid molecules include deoxyribonucleic acid (DNA) and/or ribonucleic acid (RNA). Nucleic acid molecules can be synthetic or derived from naturally occurring sources.

In one embodiment, nucleic acid molecules are isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. In certain embodiments, the nucleic acid molecules are obtained from a single cell. Biological samples for use in the present invention include viral particles or preparations. Nucleic acid molecules can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid molecules can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA. In certain embodiments, the nucleic acid molecules are bound as to other target molecules such as proteins, enzymes, substrates, antibodies, binding agents, beads, small molecules, peptides, or any other molecule and serve as a surrogate for quantifying and/or detecting the target molecule.

Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Nucleic acids may also be extracted by the UR-NA extraction procedure described herein. Nucleic acid molecules may be single-stranded, double-stranded, or double-stranded with single-stranded regions (for example, stem-and loop-structures).

### F. Nucleic Acid Extraction Buffers

Nucleic acid extraction buffers useful in the disclosed DNA extraction methods are detergent free. An example of a useful DNA and RNA extraction buffer that enables ultra-rapid nucleic acid extraction for ddPCR includes Buffer ME from the Swift X Media kit (Xpedite Diagnostics).

### III. UR-ddPCR

Droplet Digital PCR (ddPCR) is a method for performing digital PCR that is based on water-in-oil emulsion droplet technology, and it is used to amplify and quantify DNA or RNA in a sample. In ddPCR, a droplet generator separates a PCR reaction into thousands of droplets (for example, approximately 20,000 droplets when utilizing the Bio-Rad QX200 droplet generation instrument), the droplets are filled with nucleic acids and the components required for amplification (buffer, polymerase, primers, and fluorescent probes), the droplets undergo thermal cycling such that target nucleic acids are amplified in each individual droplet, a flow cytometer reads the fluorescence of each droplet, the number of positive and negative droplets is counted and the concentration of input DNA or RNA is determined based on the numbers of fluorescent droplets by Poisson distribution. For example, (A) ddPCR reactions are prepared in a tube. (B) The ddPCR reactions are partitioned into individual droplets by a droplet generator. (C) The droplets are transferred to a 96-well plate for PCR amplification. (D) Fluorescence is measured for each droplet in two fluorescent channels. A typical ddPCR protocol currently takes >2 hours to perform, not including the time for extracting DNA from tissue.

UR-ddPCR can be used in many applications including but not limited to detecting and quantifying rare mutations; detecting and quantifying nucleic acids of infectious disease agents including but not limited to viruses, bacteria, or fungi; detecting and quantifying mutations in cell-free DNA; measuring DSBs at the Cas9 cut site; diagnosing disease, for example, chronic myeloid leukemia, non-small cell lung cancer, etc.

The UR-ddPCR method optionally includes an ultra-rapid DNA/RNA extraction method (herein UR-Nucleic acid (NA) extraction) (herein, UR-NA extraction) that is compatible with ddPCR, and it includes a necessary ultra-rapid thermal cycling (UR-TC) method, the combination of which reduces the ddPCR tissue-result time by about 80%, to ˜20 mins, preferably, <20 mins. The method may also be performed for previously extracted nucleic acids with only the UR-TC procedure.

In some forms, UR-ddPCR may be performed for nucleic acids extracted rapidly by alternative methods than the UR-NA method disclosed herein, such as commercial kits (Lucigen QuickExtract and Qiagen QIAamp Fast DNA Tissue Kit), sonication, or microwave radiation. These methods may be used when any utilized detergents are removed prior to the completion of the extraction process to avoid damage to subsequent ddPCR droplet formation. A DNA extraction method using microwave is disclosed for example in U.S. Pat. No. 11,781,132 and Taglia, et al., Forensic Sci Int Synerg. 2022 5:100291 (doi: 10.1016/j.fsisyn.2022.100291), incorporated herein by reference.

### A. Ultra-rapid Nucleic Acid Extraction

The method optionally includes extracting DNA/RNA from a sample by contacting the sample with an extraction buffer free of detergent. Thus, for DNA extraction, the method includes extracting DNA from a sample using a detergent-free DNA extraction buffer. For RNA extraction, the method includes extracting RNA from a sample using a detergent-free

RNA extraction buffer. The UR-NA extraction method includes at least three steps: (i) tissue homogenization (using stainless steel beads for example) for about 30 seconds in the presence of a detergent-free buffer (for example, SwiftX Buffer ME for DNA extraction), (ii) heat incubation at about 98° C. for about 2.5 minutes, and (iii) a brief about 10-second centrifugation at setting of ˜2,000×g to separate cellular debris from clarified lysate containing nucleic acid, to provide a complete processing time about 5 minutes for one tissue sample. FIG. 1C. In a typical DNA extraction method, the extraction buffer includes detergents (for example, surfactants) that disrupt cell membranes and release DNA, and a subsequent wash step is employed to remove detergents. By contrast, by using a detergent-free DNA/RNA extraction buffer, the disclosed method eliminates steps necessary to remove detergent from extracted DNA, prior to UR-ddPCR.

In some forms the tissue sample is a human tissue sample, for example, a brain tissue sample, and the tissue is homogenized using 0.2 mm RNase-free Stainless-Steel Beads.

In some forms, the nucleic acid being extracted is DNA. The resulting UR-DNA lysate usually separates into three layers: an opaque top layer, a clear middle layer, and a bottom layer containing the beads and cellular debris. DNA is present in the two top layers. In some forms, the middle layer for UR-ddPCR minimizes damage to droplets that can occur from cellular lipids in the opaque top layer. DNA from either the clear or top opaque layers is compatible with UR-ddPCR.

In some forms, an alternative method for nucleic acid extraction may be utilized for UR-ddPCR as described above, instead of this disclosed UR-NA extraction method, as long as the extracted nucleic acid product does not damage subsequent droplet integrity, for example due to the presence of detergent.

### B. Ultra-Rapid Thermal Cycling (UR-TC)

A PCR cycle is a series of steps that repeats to make copies of a DNA sequence. The cycle consists of three main stages: denaturation, annealing, and extension. The temperature is raised to separate the double-stranded DNA into single strands in the denaturation step.

The temperature is then lowered to allow primers to bind to the target DNA in the annealing step. Finally, the temperature is raised to allow DNA polymerase to extend the primers along the template strands. In some forms, the annealing and extension steps may occur at the same temperature.

Droplet Digital PCR (ddPCR) combines microfluidics technology, PCR, and TaqMan-typeprobes to achieve precise target DNA quantification at high levels of sensitivity and specificity. The ddPCR workflow begins like any other PCR assay. DNA template material, either from a genomic or cDNA preparation, is combined with buffer, dNTPs, primers, and DNA polymerase. In addition, the ddPCR reaction may contain fluorescently labeled internal hybridization probes (TaqMan probes) for detection of the amplification product. The ddPCR reaction may also contain a non-specific DNA quantification dye, such as SYBR Green, rather than probes. The reaction is normally set up as a duplex PCR where one primer pair/probe set is targeted for a sequence of interest and a second primer pair/probe set is targeted for a reference sequence. In other cases, only one primer pair and two probes are utilized to quantify two different sequences at the same region of interest. The two probes are labeled with different fluorophores (usually FAM and VIC) and contain a fluorescence quencher, and fluorescence is detected only when the corresponding amplification product is present as the polymerase digests the probe to separate the fluorophore from the fluorescence quencher. Using microfluidic technology, the reaction mix is partitioned into thousands of tiny spherical droplets composed of an oil surface and an aqueous core containing the PCR reaction mix. For example, one 20 μl reaction may be partitioned into 20,000 reactions of ˜1 nl each. The reaction is designed such that all of the droplets will contain the standard materials (enzyme, primers, probes) but there will be a random distribution of droplets containing the target and the reference sequences of interest such that droplets will contain 0, 1, 2, or more copies of each. The PCR reaction is carried out using a standard thermal cycler. After amplification, the fluorescence of each droplet is read in succession by a droplet reader (an instrument similar to a flow cytometer). Droplets that contain the target sequence of interest or the reference sequence will fluoresce in the corresponding channel (positive droplets), while those without target will not (negative droplets)—thus the “digital” nature of ddPCR. The counts of positive and negative droplets for each target are related to the target's concentration in the sample by Poisson statistics.

The copies per droplet can be converted to the number of copies per μl by knowing the volume of reaction per droplet (˜1 nl). In this way, the absolute concentration of the target DNA (sequence of interest or reference sequence) can be calculated without using standard samples (reviewed in Maziaka, et al., Curr Protoc Hum Genet. 2014, 14;82:7.24.1-7.24.13). Poisson statistics can also be used to calculate the fraction of each of two sequence variants at the same target locus, for example, quantifying the fraction of DNA containing a mutation relative to a wild-type (non-mutant) sequence. Standard ddPCR thermal cycling includes the steps shown in the table below, reproduced from Lam, et al, https://clellandlab.ucsf.edu/sites/g/files/tkssra7856/f/wysiwyg/Protocols/PCR_ddPCR_protoc ol.pdf, Feb. 20, 2025).

| Step | | Temperature | | Time | | Ramp rate |
|---|---|---|---|---|---|---|
| 1 | Enzyme Activation | 94° | C. | 10 | mins | |
| 2 | Denaturation | 94° | C. | 30 | sec | 2° C./sec |
| 3 | Annealing Extension | *50-65° | C. | 1 | min | |
| 4 | Repeat from step 2 (39x) | | | | | |
| 5 | Enzyme Deativation | 98° | C. | 10 | mins | |

| | | | | |
|---|---|---|---|---|
| 6 | Hold | 4° | C. | ∞ |

In a standard ddPCR (droplet digital PCR) reaction, the first step typically involves heating the reaction mixture to a high temperature, usually around 95° C. for a set duration (like 10 minutes), which is done at the beginning of the PCR cycling process to activate the DNA polymerase enzyme before the amplification cycles begin; this step is often referred to as “enzyme activation” or “initial denaturation”.

In the disclosed UR-ddPCR, the cycling time of UR-ddPCR is shortened compared to standard ddPCR while maintaining significant ddPCR signal level by (a) increasing the concentration of primers, probes, and polymerase (b) eliminating the 10-minute enzyme activation step by incorporating a polymerase that is immediately activated at a temperature above room temperature but below the annealing/extension temperature, (c) reducing the denaturation time from 30 seconds to about 1 second to about 3 seconds, (d) reducing the annealing/extension step from 60 seconds to as low as about 1 second to about 10 seconds, (e) performing a total of about 35 to about 45 cycles (denaturation and annealing/extension cycles) (f) eliminating the 10-minute enzyme deactivation step (g) conducting thermal cycling with the droplets in a droplet container that has high heat thermal conductance such as a stainless-steel capillary and (h) conducting thermal cycling using a thermal cycling system that can rapidly cycle the reaction vessel between the required PCR temperature levels such as two pre-heated water baths (FIG. 2B). These selections can reduce the total thermal cycling time to as low as 3 minutes while maintaining significant ddPCR signal level. Thermal cycling is performed with a denaturation temperature from about 85° C. to about 100° C. and an annealing/extension temperature from about 50° C. to about 78° C.

Thus, UR-TC as used herein in connection with the disclosed UR-ddPCR refers to thermal cycling in which denaturation step lasts from about 1 to about 3 seconds and the sum of each UR-TC PCR cycle's annealing and extension steps (or each PCR cycle's annealing/extension step when annealing and extension are combined in one step) lasts from about 1 second to about 10 seconds.

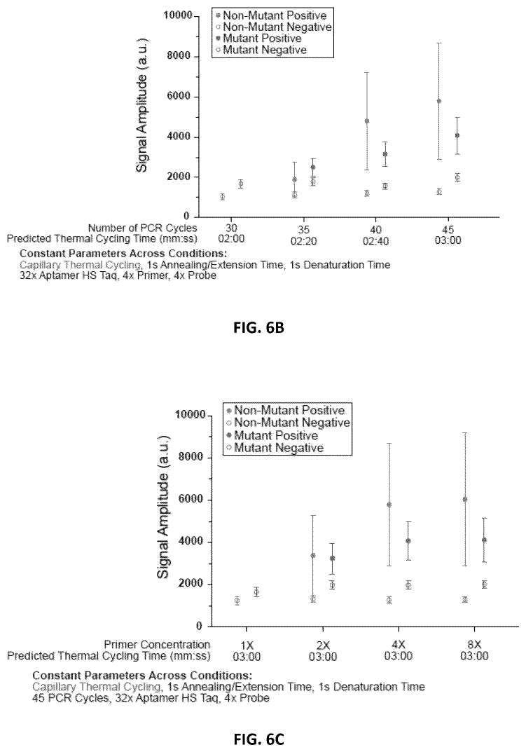

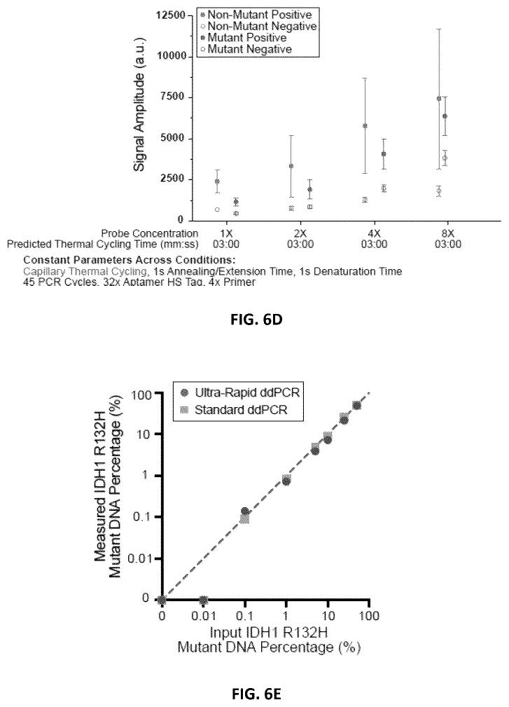

As demonstrated in the Examples, reducing the annealing/extension step from 60 seconds to 1 second is accomplished by adding a rapidly activating polymerase such as the Aptamer HS Taq polymerase (NEB) at a concentration that is about 25-fold more than is required for standard PCR, and increasing the concentrations of primers and probes to about 4-fold the amount required for standard ddPCR (FIG. 2D and FIGS. 6B-D). This yielded an unprecedented total ddPCR thermal cycling time of 3 minutes (FIG. 2E), which meets the time constraints necessary for a tissue-to-result UR-ddPCR assay applicable to critical care settings such as the operating room. For each target nucleic acid sequence the user wishes to detect, the concentration of polymerase, primers, and probes required to achieve UR-ddPCR may be determined using a series of reactions, each of which contains a positive control input DNA and increasing amounts of primer, probe, and polymerase (for example 2-fold, 4-fold, 8-fold, etc. relative to standard ddPCR) and then performing UR-TC to determine which minimal reagent concentration combination produces a separation of fluorescence signal between positive and negative droplets.

Prior to UR-TC, an UR-DNA sample is contacted with a UR-ddPCR reaction mix. Subsequently, droplets are formed using an appropriate droplet generation oil by a microfluidic droplet generator.

The UR-TC method includes the steps of: (a) contacting an UR-DNA sample with an aptamer-inhibited hot-start Taq polymerase and primers configured for amplification of the target nucleic acid sequence, wherein the polymerase is provided at a final reaction concentration from about 0.15 U/μL to about 1 U/μL (6× to 40×, where 1× concentration is 0.025 U/μL); primers are each provided at a final reaction concentration of at least 1.8 μM to 10.8 UM each (2× to 12×, where 1× concentration is 0.9 μM for each primer); and probes at a final reaction concentration from about 0.375 μM to about 3 μM (1.5× to 12×, where 1× concentration is 0.25 μM for each probe), (b) generating droplets including the polymerase, primer and target nucleic acid sequence and (c) amplifying the target nucleic acid sequence by thermally cycling the UR-DNA sample between at least a denaturation temperature and an annealing/extension temperature through a plurality of amplification cycles using where each cycle is completed in about 2 to less than about 13 seconds, per cycle, for example, in about 2, 3, 4, 5, 6 7, 8, 9, 10, 11, 12, or 13 seconds per cycle wherein the droplets are loaded for thermal cycling into a droplet container with high thermal conductance such as thin-walled stainless steel capillaries, wherein the plurality of cycles is at least 30, 35, 40, 45 or 50 cycles. For example, denaturation can be completed between 1 to 3 seconds, and annealing/extension, between 1 to about 10 seconds. In some forms, thermal cycling is performed by contacting the droplet-loaded capillaries with a water bath at 95° C., for about 1second for denaturation and contacting the droplet-loaded capillaries with a water bath at 62° C. for about 1 to about 5 seconds of annealing/extension, in some forms, for at least 30, 35,40, 45 or 50 cycles.

In some forms, each cycle is completed in less than 11 seconds.

In some forms, each cycle is completed in less than 9 seconds.

In some forms, each cycle is completed in less than 7 seconds.

In some forms, each cycle is completed in less than 5 seconds.

In some forms, each cycle is completed in less than 3 seconds.

In an exemplary method disclosed herein, for each sample, a 21.5 μL of UR-DNA extract reaction mix is prepared, containing: a) 1X ddPCR Supermix for Probes, no dUTP, b) 3.6 μM of each forward and reverse primer, c) 1 μM of each non-mutant and mutant targeting probe, d) 0.23 U/μL of HaeIII, restriction enzyme and e) from about 0.625 U/μL to about 0.8 U/μL Hot Start (aptamer-based) Taq DNA Polymerase (NEB). Primer and probe concentrations are 4× the concentrations used in standard ddPCR, and the Aptamer HS Taq polymerase is 32×(0.8 U/μL) or 25X (0.625 U/μL) relative to the 0.025 U/μL concentration recommended by NEB for standard PCR.

0.5 μL of UR-DNA extract is then added to the UR-ddPCR reaction mix.

Droplets are generated from the UR-DNA-ddPCR reaction mix using a microfluidic droplet generator and an appropriate droplet generation oil, and using methods known to one of ordinary skill in the art, including commercially available droplet generation systems, for example, QX200 Droplet Generator (Bio-Rad). Once final droplets have been produced by any of the droplet forming method, the droplets are thermal cycled, resulting in amplification of the target nucleic acid in each droplet. Droplet based digital PCR technology, as described in Link et al. (U.S. patent application numbers 2008/0014589, 2008/0003142, and 2010/0137163), Anderson et al. (U.S. Pat. No. 7,041,481 and which reissued as U.S. Pat. No. RE 41,780) and European publication number EP2047910 to Raindance Technologies Inc, (the contents of each of which are incorporated by reference herein in their entireties) utilizes a single primer pair per library droplet. This library droplet is merged with a template droplet which contains all the PCR reagents including genomic DNA except for the primers. After merging of the template and the primer library droplets the new droplet now contains all the reagents necessary to perform PCR. The droplet is then thermal cycled to produce amplicons.

However, in ultra-rapid ddPCR, after generating droplets, instead of loading the droplets into a 96-well plate, the droplets are loaded into a droplet container that has high thermal conductance, such as a thin-walled stainless steel capillary, which is used for thermal cycling.

In some forms, droplets are loaded into the capillaries by loading a 200 μL pipette set to 45 μL with a pipette tip (Corning 4138). In this process, the pipette is loaded with a tip, then the capillary is manually inserted into the open end of the loaded pipette tip. The opposite end of the capillary not attached to the pipette tip is then put into the droplet generation cartridge at approximately a 45° C. angle and used to aspirate the droplets. The end of the capillary not attached to the pipette tip was then capped using a temperature-resistant silicone cap (92805K3, McMaster-Carr) that was pre-cut to 4 mm in length. Specifically, the cap is placed on the capillary by holding the pipette in one hand, then placing the capillary, while still attached to the pipette tip, between the middle and ring fingers of the other hand for support and carefully sliding the cap onto the tip of the capillary using the thumb and index fingers. The capillary is then gently detached from the pipette tip by holding the pipette vertically so that the capped end of the capillary was pointed downwards and pulling lightly from the middle of the capillary with one hand while holding the pipette in the other hand. The now exposed end of the capillary that was previously attached to the pipette tip and now facing upwards is then gently capped with another pre-cut 4 mm silicone cap. Finally, both silicone caps on either end of the capillary were gently squeezed towards each other to ensure a tight seal. FIG. 2B.

The capillary loaded droplets are rapidly thermal cycled by placing the capped capillaries in a steel wire holder and manually moving the capillaries between the two adjacent pre-heated water baths for an effective number of cycles. As exemplified herein, for the IDH R132H assay, 45 cycles of denaturation (95° C. water bath) are performed and 1 second of annealing/extension (62° C. water bath) is performed; for the BRAF V600E assay, 40 cycles of denaturation (95°° C. water bath) and 5 seconds of annealing/extension (62° C. water bath) are performed.

After thermal cycling, the droplets are subjected to a detection step, for detection of amplification products. Generally, droplets are flowed to a detection module for detection of amplification products. The detection module is in communication with one or more detection apparatuses. The detection apparatuses can be optical or electrical detectors or combinations thereof. Examples of suitable detection apparatuses include optical waveguides, microscopes, diodes, light stimulating devices, (e.g., lasers), photo multiplier tubes, and processors (e.g., computers and software), and combinations thereof, which cooperate to detect a signal representative of a characteristic, marker, or reporter, and to determine and direct the measurement or the sorting action at a sorting module. Further description of detection modules and methods of detecting amplification products in droplets are shown in Link et al. (U.S. patent application numbers 2008/0014589, 2008/0003142, and 2010/0137163).

Amplified targets can be detected using detectably labeled probes.

In some forms, the capillary contents are then slowly dispensed into a ddPCR 96-well plate well. This unsealed ddPCR 96-well plate containing the amplified droplets is placed directly in the QX200 Droplet Reader and the amplified droplets are then read. In some forms, UR-ddPCR is used to detect a mutation in a target nucleic acid. The mutation is preferably a cancer hotspot mutation.

Mutational hotspots indicate selective pressure across a population of tumor samples.

Hotspot mutations in cancer genes are reviewed in Chang, et al., Nat Biotechnol. 34:155-163 (2016) and Chang, et al., Cancer Discov (2018) 8 (2): 174-183 (the specific examples of which are incorporated herein by reference), non-limiting examples of which are, IDH1 R132H, and BRAF V600E. Additional cancer hotspots are shown in the Table below (reproduced from Chang, et al., Nat Biotechnol. 34:155-163 (2016)).

| Symbol | Position | Tumor types |
|---|---|---|
| H3F3A | K27M | High-grade pontine glioma (26) |
| | | Pilocytic astrocytoma (1) |
| MYD88 | L273P | Chronic lymphocytic leukemia (12) |
| | | Non-hodgkin lymphoma (2) |
| | | Multiple myeloma (1) |
| STK19 | D89N | Cutaneous melanoma (13) |
| | | Squamous cell carcinoma (5) |
| EGFR | G598V/A | Glioblastoma (15) |
| | | Low-grade glioma (3) |
| | | High-grade pontine glioma (1) |
| PPP2R1A | P179R/L | Endometrial (8) |
| | | Uterine carcinosarcoma (5) |
| FGFR3 | Y375C | Urothelial bladder (7) |
| | | Renal papillary cell carcinoma (1) |
| KNSTRN | S24F | Cutaneous melanoma (11) |
| | | Squamous cell carcinoma (2) |
| CCND1 | Y44D/S/H/F/C/* | Mantel cell lymphoma (6) |
| | | Multiple myeloma (1) |
| CRNKL1 | S128F | Cutaneous melanoma (8) |
| | | Squamous cell carcinoma (2) |
| EGFR | L861Q | Lung adenocarcinoma (5) |
| | | Lung squamous cell carcinoma (2) |

Additional cancer hotspot mutations are known in the art (Chang, et al., Nat Biotechnol. 34:155-163 (2016).

The examples herein demonstrate identification of IDH1 R132H and BRAF V600E cancer hotspot mutations that are present in many low-grade gliomas and melanomas, respectively.

In some forms, detection of IDH1 R132H or BRAF V600E clonal mutation are performed intraoperatively, and their intraoperative detection can be used shape surgical decision-making.

IDH1 R132H is a mutation in the IDHI gene that occurs in many gliomas. It is a point mutation that occurs in the isocitrate dehydrogenase 1 (IDH1) enzyme. IDH1 R132H mutation is an important marker of tumor subtype that correlates with survival in patients with gliomas. Current treatment methods for patients with glioma include surgical resection, radiation therapy, targeted IDH1 inhibition, and chemotherapy with the use of temozolomide (TMZ), yet the long term survival rate remains poor. Studies indicate brain tumors harboring the IDH1 mutation offer a median overall survival of 3.8 years compared to 1 year for patients without an IDH1 mutation. Improved survival for tumors with an IDH1 mutation is also associated with more maximal surgical resection.

BRAF V600E is a specific genetic mutation in the BRAF gene, which encodes a protein involved in cell growth and division. The V600E mutation causes a change in the amino acid sequence of the BRAF protein at position 600, from valine (V) to glutamic acid (E). This change alters the protein's function, leading to uncontrolled cell growth and the development of cancer. The cancers associated with the BRAF gene mutation are not specific to one part of the body or a certain cell type. These cancers include: melanoma (about half of all melanomas have the BRAF gene mutation), Hairy cell leukemia, non-Hodgkin lymphoma, thyroid cancer, ovarian cancer, lung adenocarcinoma, colorectal cancer, certain brain cancers, including papillary craniopharyngioma, pilocytic astrocytoma, and pediatric low-grade glioma.

Surgeons in the operating room often do not have information about the genetic mutations present in the tumor they are removing. A technology for very rapid and accurate quantification of mutations in the operating room would allow surgeons to tailor surgery to the cancer's genetic profile, and it would allow surgeons to more reliably remove tumor tissue.

The technology and workflow developed here enables intraoperative molecular-genetic assays with unprecedented speed and sensitivity, providing real time knowledge of mutation status during surgery

The disclosed methods, compositions and devices will be further understood in view of the following non-limiting examples.

### EXAMPLES

### Materials and Methods

### Experimental Model and Study Participant Details

### Tissues for Laboratory Experiments

Laboratory experiments were conducted using tissues from three surgical resections of IDH1 R132H oligodendrogliomas of subjects who provided consent under a human subjects protocol approved by the New York University Grossman School of Medicine Institutional Review Board. These three tumors were confirmed by clinical sequencing to have the IDH1 R132H mutation.

Healthy cerebral cortex tissue (i.e., without the IDH1 R132H mutation) was obtained from the NIH NeuroBioBank (subject ID 5606, Broadman's area 20).

### Intraoperative UR-ddPCR

All patients whose samples were profiled as part of intraoperative studies provided consent under a human subjects protocol approved by the New York University Grossman School of Medicine Institutional Review Board. Subject information on sex and age was collected by the clinical team. Information on race, gender, and socioeconomic status was not collected.

### Method Details

### Processing Tissues for Laboratory Experiments

One tumor sample was frozen fresh and initially stored at −80° C., followed by standard DNA extraction (see below protocol) for use as positive control DNA for the IDH1 R132H standard ddPCR and UR-ddPCR laboratory experiments. The two other tumor samples were cut into small pieces (˜3×3×3 mm), each placed into separate 2 mL DNA LoBind tubes (Eppendorf), frozen fresh, and stored at −80° C. for later use in the tissue-to-result UR-ddPCR laboratory experiments.

Healthy cerebral cortex tissue was thawed, cut into small pieces (˜3×3×3 mm), placed into 2 mL DNA LoBind tubes, and frozen again at −80° C. for use as a negative control for the laboratory tissue-to-result UR-ddPCR experiments.

Standard DNA Extraction from Tissues

Standard DNA extractions were conducted using the QIAamp DNA Mini Kit (Qiagen) per the manufacturer's “DNA Purification from Tissues” protocol, including the optional RNase digestion step. DNA was eluted in 100 μL of 10 mM Tris pH 8 and stored at −20° C. DNA quality was assessed with the NanoDrop One instrument (ThermoFisher) and quantified with the Qubit 1X dsDNA High Sensitivity Assay Kit (ThermoFisher).

ddPCR assay design

IDH1 R132H and BRAF V600E ddPCR assay primers were designed with a combination of Primer335 and manual design; probes (5′ HEX or FAM fluorophores with 3′ Iowa Black quenchers) were designed by Integrated DNA Technologies (IDT). Primer and probe sequences are listed in Table 1 and 2.

TABLE 1
ddPCR assay primers

| | | Coordinates | | | | | |
|---|---|---|---|---|---|---|---|
| ddPCR | | (hg38) | | Amplicon | | | Primer |
| Assay | Amplicon | Target | | Coordinates | Primer | Primer | Tm |
| Name | Size | Mutation | Mutation | (hg38) | Direction | Sequence | (C) |
| IDH1 | 71 | chr2: | G > A | chr2: | Forward | CTTGTGAGTGGATGGGTAAA | 65.1 |
| R132H | | 208,248,388 | | 208248355- | | ACCTA (SEQ ID NO: 1) | |
| | | | | 208248425 | | | |
| IDH1 | 71 | chr2: | G > A | chr2: | Reverse | CCAACATGACTTACTTGATC | 65.1 |
| R132H | | 8208,24,388 | | 208248355- | | CCCATA (SEQ ID NO: 2) | |
| | | | | 208248425 | | | |
| BRAF | 132 | chr7: | T > A | chr7: | Forward | CCAGACAACTGTTCAAACTG | 66.0 |
| V600E | | 140,753,336 | | 140753291- | | ATGGG (SEQ ID NO: 3) | |
| | | | | 140753422 | | | |
| BRAF | 132 | chr7: | T > A | chr7: | Reverse | ACTGTTTTCCTTTACTTACTA | 61.3 |
| V600E | | 140,753,336 | | 140753291- | | CACCTCAGA (SEQ ID NO: 4) | |
| | | | | 140753422 | | | |

TABLE 2
ddPCR assay probes

| | | Target | | | | Probe | Probe |
|---|---|---|---|---|---|---|---|
| | | Mutation | | | Probe | Match | Mismatch |
| | Target | Coordinates | | Fluorophore/ | Sequence | Tm | Tm |
| t | Genotype | (hg38) | Mutation | Quencher | (5′ −> 3′) | (C.) | (C.) |
| IDH1 | Non- | chr2: 208, | G > A | 5′ HEX/3′ | TG + A + C + | 65.7 | 48.6 |
| R132H | Mutant | 248, 388 | | Iowa Black | GA + C + CTATG | | |
| | | | | | (SEQ ID NO: 5) | | |
| IDH1 | Mutant | chr2: 208, | G > A | 5′ FAM/3′ | TG + A + T + | 65.8 | 54.4 |
| R132H | | 248, 388 | | Iowa Black | GA + C + CTA + | | |
| | | | | | TG (SEQ ID NO: 6) | | |
| BRAF | Non- | chr7: 140, | T > A | 5′ HEX/3′ | AGCTA + C + AG + | 65.2 | 55.5 |
| V600E | Mutant | 753, 336 | | Iowa Black | T + G + AAAT | | |
| | | | | | (SEQ ID NO: 7) | | |
| BRAF | Mutant | chr7: 140, | T > A | 5′ FAM/3′ | AGCTA + C + AG + | 63.8 | 54.7 |
| V600E | | 753, 336 | | Iowa Black | A + G + AAAT | | |
| | | | | | (SEQ ID NO: 8) | | |

′+ indicates PrimeTime locked nucleic acids (IDT)

The primers were designed to have short amplicons (<150 base pairs) to reduce the time necessary for the polymerase to replicate the amplicon-thereby lowering the required annealing/extension time in PCR. Importantly, these studies showed that in UR-ddPCR, PrimeTime locked nucleic acid probes (IDT) perform better than Affinity Plus probes (IDT) (FIG. 7), so the former were used in experiments. Probes were designed with locked nucleic acids to enable shorter probes, which in turn increases the difference in melting temperatures for a matched versus mismatched base at the mutation location.

Testing rapid DNA extraction buffer compatibility with ddPCR droplets

The compatibility of rapid DNA extraction buffers (which may contain droplet-damaging detergents) with ddPCR droplets was tested, before developing the ultra-rapid DNA extraction. The following three buffers were tested: Buffer DL from the SwiftX DNA extraction kit (Xpedite Diagnostics), QuickExtract DNA Extraction Solution (Lucigen), and Buffer ME from the Swift X Media kit (Xpedite Diagnostics).

4 μL of each rapid DNA extraction buffer was added to a standard ddPCR mix with a final volume of 22 μL containing: a) 1X ddPCR Supermix for Probes, no dUTP (Bio-Rad), b) 0.9 μM of each forward and reverse primer (IDT), c) 0.25 μM of each non-mutant and mutant probe (IDT), d) 0.23 U/μL of HaeIII (NEB). Standard droplet generation was conducted as described in the Standard ddPCR in the laboratory methods section, however, instead of adding the droplets to a plate, 10 μL of the extract was added to a hemocytometer, covered with a coverslip, and imaged with light microscopy to determine the buffer's compatibility with ddPCR droplets.

### Ultra-Rapid DNA Extraction in the Laboratory

Ultra-rapid (UR) DNA extraction-the first step of tissue-to-result UR-ddPCR-was conducted for up to four samples in parallel.

To maximize speed, prior to the protocol the following was prepared: 1. Buffer ME was activated with Component P (Xpedite Diagnostics) by adding one 1 mL of Buffer ME to the tube containing dry Component P, pipetting thoroughly, transferring the full volume of resuspended Component P back into the Buffer ME bottle, mixing by inversion, and then aliquoting and storing the reagent at −20° C.; 2. The TissueLyser II (QIAGEN) instrument was set to a 30 Hz, 30 second program; 3. A ThermoMixer C (Eppendorf) was preheated to 98° C.; and, 4. For each sample, 0.1 grams of 0.2 mm RNase-free Stainless-Steel Beads (Next Advance) was pre-measured into 0.2 mL PCR tubes.

After completing these preparation steps, 200 μL of activated Buffer ME and the 0.1 grams of pre-measured 0.2 mm RNase-free Stainless-Steel Beads were added to the 2 mL DNA LoBind Tube containing the tumor tissue. Each sample was homogenized with the TissueLyser II at 30 Hz for 30 seconds. Then, the post-homogenization mixture was incubated at 98° C. for 2.5 minutes on a ThermoMixer C to extract the DNA. Finally, this post-incubation lysate was briefly centrifuged for 10 seconds in an LSE Mini Microcentrifuge (Corning) to help separate cellular debris form layers containing the DNA used in ddPCR.

The resulting UR-DNA lysate usually separates into three layers: an opaque top layer, a clear middle layer, and a bottom layer containing the beads and cellular debris. DNA is present in the two top layers; when the clear middle layer has sufficient volume and is clearly distinct from the top opaque layer, the middle layer is used for UR-ddPCR to minimize possible damage to subsequent droplets that can occur from cellular lipids in the opaque top layer. When the clear middle layer has insufficient volume or is not clearly distinct from the top opaque layer, the top opaque layer is used for UR-ddPCR. While the clear middle layer is preferable, DNA from either the clear or top opaque layers is compatible with UR-ddPCR.

Standard ddPCR in the Laboratory

The following protocol was used for standard ddPCR for both IDH1 R132H and BRAF V600E mutations:

For each sample, either 30 ng of genomic DNA or up to 0.5 μL of UR-DNA extract was added to a ddPCR mix with a final volume of 22 μL containing: a) 1X ddPCR Supermix for Probes, no dUTP (Bio-Rad), b) 0.9 μM of each forward and reverse primer (IDT), c) 0.25 μM of each non-mutant and mutant probe (IDT), and, d) 0.23 U/μL of HaeIII (NEB). HaeIII is a type II restriction endonuclease that cuts double-stranded DNA but not within the DNA sequence targeted by these assays. HaeIII reduces the average size of DNA fragments to improve their random incorporation into droplets. HaeIII recognizes the sequence 5′-GGCC-3′ and cuts after the G. 20 μL of this ddPCR mix was added to a DG8 droplet generation cartridge (Bio-Rad) sample well. 20 μL of 1X ddPCR Buffer Control for Probes (Bio-Rad) was then added to all unused DG8 cartridge sample wells and 70 μL of Droplet Generation Oil for Probes (Bio-Rad) to all DG8 cartridge oil wells. The DG8 cartridge was covered in a rubber gasket (Bio-Rad) and loaded in a QX200 Droplet Generator (Bio-Rad) to generate droplets. After generating droplets, 40 μL of the droplets was transferred from each sample to a ddPCR 96-well Plate (Bio-Rad, Cat. 12001925) and the plate was sealed with a Pierceable Foil Heat Seal (Bio-Rad) and a PX1 PCR Plate Sealer (Bio-Rad).

The sealed ddPCR droplets were then thermal cycled on an Eppendorf Mastercycler X50L with the following protocol: 95° C. for 10 minutes, 45 cycles (IDH1 R132H) or 40 cycles (BRAF V600E) of 95° C. for 30 seconds and 62° C. for 1 minute, and 98° C. for 10 minutes. We read the amplified ddPCR droplets on a QX200 Droplet Reader (Bio-Rad).

Ultra-Rapid ddPCR in the Laboratory

Ultra-rapid (UR) ddPCR was developed via experiments that with design considerations including, a selection of reagent concentrations and thermal cycling method, as detailed in the main text and figures. UR-ddPCR experiments (FIGS. 2A-D and FIGS. 6A-6D) utilized DNA extracted from an IDH1 R132H-mutant tumor as described in the ‘Tissues for laboratory experiments’ section. The final UR-ddPCR protocol, which is compatible with UR-DNA extraction, is described here.

Prior to performing UR-ddPCR, the following was prepared:

1. Two 1-liter water baths, one at 95° C. and one at 62° C. The water baths were heated on hot plates with continuously active magnetic stir bars, and temperatures were continuously measured with standard thermometers.

2. For each sample, a 20 μL (or 21.5 μL, if UR-DNA extract is assayed) reaction mix was prepared containing: a) 1X ddPCR Supermix for Probes, no dUTP, b) 3.6 μM of each forward and reverse primer, c) 1 μM of each non-mutant and mutant probe, d) 0.23 U/μL of HaeIII, and e) 0.8 U/μL (all laboratory UR-ddPCR experiments except for one of the IDH1 R132H ddPCR sensitivity experiments and the BRAF V600E sensitivity experiment) or 0.625 U/μL of Hot Start (aptamer-based) Taq DNA Polymerase (taken from a 20 U/μL stock that was made by diluting a 100 U/μL high-concentration preparation of polymerase custom ordered, catalog #M0495B-HC3, from NEB with 1× standard Taq buffer from NEB). As described in the text, the above primer and probe concentrations are 4× the concentrations used in standard ddPCR, and the Aptamer HS Taq polymerase is 32× (0.8 U/μL) or 25X (0.625 U/μL) relative to the 0.025 U/μL concentration recommended by NEB for standard PCR.

3. 20 μL of 1X ddPCR Buffer Control for Probes was added to all unused DG8 cartridge wells and 70 μL of Droplet Generation Oil for Probes (Bio-Rad) to all DG8 cartridge oil wells.

4. The QX Manager 2.1 Software (Bio-rad) was initialized with the plate name, probes, and selected wells for analysis.

After completing these preparation steps, 2 μL of 15 ng/μL genomic DNA or 0.5 μL of UR-DNA extract was added to the reaction mix. ddPCR droplets were then generated as described above for standard ddPCR. However, after generating droplets, instead of loading the droplets into a 96-well plate, the droplets were loaded into thin-walled stainless steel capillaries (obtained from either Ziggy's Tubes and Wires (most experiments; product 18X304-CUT) or from Component Supply Company (product HTX-18X))) with the following properties: 304 stainless steel, 18 gauge extra thin wall hypodermic tubing×2.25 inches long, 0.0495 to 0.0505 inches outer diameter, 0.041 to 0.043 inches inner diameter, welded and drawn, burr-free. Note: some manufacturers wash capillaries with detergents that can damage droplets, so capillaries are preferably additionally washed using ethanol. The droplets were loaded into the capillaries by loading a 200 μL pipette set to 45 μL with a pipette tip (Corning 4138). In this process, the pipette was loaded with a tip, then the capillary was manually inserted into the open end of the loaded pipette tip. The opposite end of the capillary not attached to the pipette tip was then put into the droplet generation cartridge at approximately a 45° C. angle and used to aspirate the droplets. The end of the capillary not attached to the pipette tip was then capped using a temperature-resistant silicone cap (92805K3, McMaster-Carr) that was pre-cut to 4 mm in length. Specifically, the cap was placed on the capillary by holding the pipette in one hand, then placing the capillary, while still attached to the pipette tip, between the middle and ring fingers of the other hand for support and carefully sliding the cap onto the tip of the capillary using the thumb and index fingers. The capillary was then gently detached from the pipette tip by holding the pipette vertically so that the capped end of the capillary was pointed downwards and pulling lightly from the middle of the capillary with one hand while holding the pipette in the other hand. The now exposed end of the capillary that was previously attached to the pipette tip and now facing upwards was then gently capped with another pre-cut 4 mm silicone cap. Finally, both silicone caps on either end of the capillary were gently squeezed towards each other to ensure a tight seal.

The ddPCR droplets were rapidly thermal cycled by placing the capped capillaries in a steel wire holder and manually moving the capillaries between the two adjacent pre-heated water baths. For the IDH R132H assay, 45 cycles of denaturation (95° C. water bath) for 1 second and annealing/extension (62° C. water bath) for one second were performed. For the BRAF V600E assay, 40 cycles of denaturation (95° C. water bath) for 1 second and annealing/extension (62° C. water bath) for 5 seconds were performed. A timer was used to determine when to switch between water baths. After thermal cycling, the capillary was held vertically and the cap facing upwards carefully removed by holding the capillary with one hand and pulling the cap with the other. The now uncapped end of the capillary was then attached to a new tip on a 200 μL pipette set to 47 μL. The silicone cap still attached to the capillary was then carefully removed by using one hand to apply pressure against the capillary towards the pipette tip, ensuring the seal is secure, holding the capillary between the index and middle finger, and pushing the cap off the capillary using the thumb while the pipette is held with the other hand. The capillary contents were then slowly dispensed into a ddPCR 96-well plate well. This unsealed ddPCR 96-well plate containing the amplified droplets was placed directly in the QX200 Droplet Reader and the amplified droplets were then read.

### Comparison of Standard and Ultra-Rapid Ddpcr Thermal Conductance and Surface Area to Volume Ratios

The thermal conductances of the ddPCR 96-well plate well used in standard ddPCR and the stainless-steel capillary used in ultra-rapid ddPCR were estimated. Thermal conductance, C, is the rate at which a material of a given thickness conducts heat. It is calculated as C=K/T and measured in Watts/(meter²*Kelvin), i.e., W/(m²*K), where K is the thermal conductivity (a heat transfer constant specific to a given material) measured in Watts/(meter*Kelvin) and T is the thickness of the material, measured in meters.

The thermal conductivity of polypropylene, the plastic used in the ddPCR 96-well plates, has been measured to be 0.22 W/(m*K) 36, and the thermal conductivity of stainless-steel 304, the material of our capillaries, is approximately 15 W/(m*K) 37. Since the thickness of the ddPCR plate wells was not provided, the thickness of 33 wells of a ddPCR 96-well plate at their thinnest point was measured using a micrometer and an estimate of 0.30+0.07 mm was obtained. The thickness of the stainless-steel capillary is 0.10+0.04 mm per the manufacturer. Thus, the thermal conductance of the ddPCR plate well is approximately 7.3×102 W/(m²*K) and the thermal conductance for the stainless-steel capillary is approximately 1.5×105 W/(m²*K), which is an ˜204-fold difference.

The surface area to volume ratio of a standard ddPCR reaction occurring in a ddPCR 96-well plate and an ultra-rapid ddPCR reaction occurring in a stainless steel capillary, was also estimated. The volume of the slanted portion of an individual well of a ddPCR 96-well plate was estimated as a truncated cone using bottom and top diameters of 2.16 mm and 5.60 mm, respectively, and a height of 11.10 mm as estimated from the manufacturer's blueprint, which yielded a volume of 139.84 μL. Using these values, the surface area of the slanted portion of the ddPCR plate well was also calculated to be 136.92 mm². Thus, the surface area to volume ratio of a standard ddPCR well is 0.98 mm²/μL. For the stainless-steel capillary, its volume was calculated as a cylinder with length 57.15 mm and an inner diameter of 1.067 mm (per the manufacturer), which yielded 51.10 μL. Using these values, the surface area of the capillary that contacts the ddPCR droplets during ultra-rapid ddPCR was also calculated to be 191.57 mm². Thus, the surface area to volume ratio of the capillary is 3.75 mm²/μL, which is 3.8-fold higher than that of the ddPCR plate well. ddPCR sensitivity experiments

To determine the sensitivities of the ddPCR assays, IDH1 R132H and BRAF V600E 50% mutant DNA percentage (heterozygous, clonal) reference genomic DNA standards (Horizon Discovery) were serially diluted with non-mutant genomic DNA (NA12878 and NA12877 [Coriell], respectively) to create mutant DNA percentages of 25%, 10%, 5%, 1%, 0.1%, and 0.01%. The mutant DNA percentages of the 50% reference DNA and each dilution was then measured using standard ddPCR and UR-ddPCR. This experiment was performed twice for the IDH1 R132H assay, once with 32X Aptamer HS Taq concentration and once with 25X concentration. This experiment was performed for the BRAF V600E assay with 25X Aptamer HS Taq concentration.

Tissue-to-Result Ultra-Rapid ddPCR Laboratory Experiments

The tissue-to-result UR-ddPCR laboratory experiments aimed to achieve the fastest possible measurement of mutant DNA percentage and to confirm that UR-ddPCR measures mutant DNA percentages comparable to those measured by standard ddPCR.

Once the previously described preparation steps for UR-DNA extraction and UR-ddPCR were completed, timers were set, and either 1 or 4 pre-aliquoted and thawed IDH1 R132H mutant tumor tissue samples were processed per the previously described UR-DNA extraction protocol. Once the DNA was extracted, the timers were paused while 10 μL of the UR-DNA extract from each sample was aliquoted to a separate PCR tube stored on ice for later use in the standard ddPCR experiment. The timers were then resumed and the UR-ddPCR assay was conducted as previously described, stopping the timers once the results were accessible on the QX200 droplet reader.

Throughout the tissue-to-result UR-ddPCR experiment in the laboratory, the total time was measured on one timer and the times for each individual step of the process were measured using other separate timers that were present at each station (DNA extraction, droplet generation, droplet loading, PCR thermal cycling, droplet dispensing, and droplet reading). The time difference between the total time and the summed step-by-step times was the considered “movement time” and was excluded from the reported “tissue-to-result total time” since it is 1) dependent on the laboratory space in which the protocol is conducted, 2) could be reduced to 30 seconds or less if all machines were present at a single location, and 3) was only 1 minute and 47 seconds on average within our unoptimized laboratory space.

Immediately following each of these tissue-to-result UR-ddPCR experiments, a standard ddPCR assay was conducted as previously described for the same samples, with an input of 0.5 μL of the UR-DNA extract that was set aside after UR-DNA extraction. This blinded standard ddPCR measurement on the same DNA extract provided an assessment of the accuracy of our tissue-to-result UR-ddPCR.

### Tissue-to-Result UR-ddPCR in the Operating Room

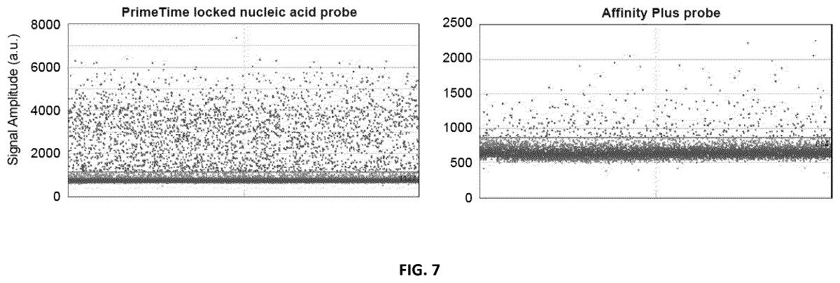

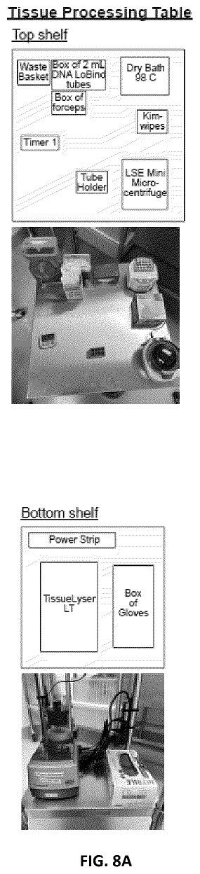

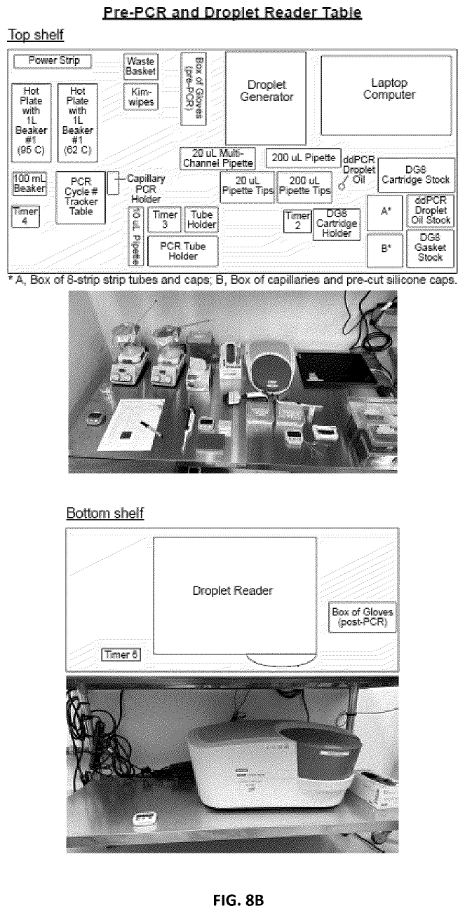

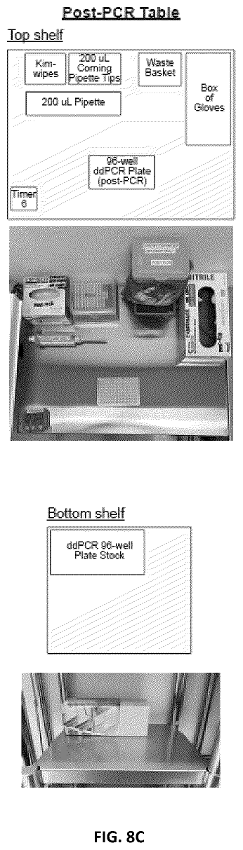

Three mobile carts were assembled for the operating room: one for UR-DNA extraction, one for pre-PCR UR-ddPCR steps, and one for post-PCR UR-ddPCR steps. The layout and all components used in each of these carts are detailed in FIG. 8A-8C and Table 3.

TABLE 3
Items required for intraoperative tissue-to-result UR-ddPCR

| Item | Manufacturer | Catalog # | # of items | |
|---|---|---|---|---|
| Cart 1: Tissue | | | | |
| Processing Table | | | | |
| Cart (24″ × 24″) | DuraSteel | SS-DR2424H | 1 | |
| 1.5 mL DNA lobind | Eppendorf | 22431021 | 1 bag (50 | |
| tubes | | | tubes) | |
| 1.5/2 mL tube | Chang | MiniR1D5ML | 2 | |
| holder | Bioscience | | | |
| Dry bath | Fisherbrand | 14-955-218 | 1 | |
| 2 mL dry bath | Fisherbrand | 14-955-225 | 1 | |
| adaptor | | | | |
| LSE mini | Corning | 6770 | 1 | |
| microcentrifuge | | | | |
| Disposable foreceps | TWD Scientific | DF8988PS | 1 box (100 | |
| | LLC | | forceps) | |
| Timer | LinkDM | LinkDm- | 1 | |
| | | E6-NEW | | |
| Waste basket | Bel-Art | F131930500 | 1 | |
| Kimwipes | Kimberly-Clark | 34155 | 1 | box |
| | Professional | | | |
| Gloves | Ansell | 6034301 | 1 | box |
| TissueLyser LT | Qiagen | 85600 | 1 | |
| TissueLyser 12-tube | Qiagen | 69980 | 2 | |
| adaptor | | | | |
| Power strip | GE | 45192 | 1 | |
| Cart 2: Pre-PCR | | | | |
| and Droplet | | | | |
| Reader Table | | | | |
| Cart (60″ × 30″) | DuraSteel | SS-DR3060H | 1 | |
| Hot plate | Fisherbrand | SP88854200 | 2 | |
| 1 L Beaker | DWK Life | 14000P1000 | 2 | |
| | Sciences | | | |
| 100 mL beaker | Corning | 1000-100 | 1 | |
| Stir bars | FisherBrand | 14-513-51SIX | 2 | |
| Thermometer | Bel-Art | B60800-2500 | 2 | |
| Thermometer holder | CAPPEC | SIC01 | 2 | |
| Capillary | Ziggy's Wires | See Methods for | 10/OR | Case |
| | and Tubes | specification | | |
| Silicone Caps | McMaster-Carr | 659177 (pre- | 20/OR | Case |
| | | cut, see | | |
| | | Methods) | | |
| ddPCR droplet Oil | BioRad | 1863005 | 1 box | |
| | | | (10 × 7 mL | |
| | | | bottles) | |
| DG8 cartidge stock | BioRad | 1864008 | 1 box (24 | |
| | | | cartidges) | |
| DG8 cartidge holder | BioRad | 1863051 | 1 | |
| DG8 gasket stock | BioRad | 1863009 | 1 bag (24 | |
| | | | gaskets) | |
| 200 uL pipette | Eppendorf | 3123000055 | 1 | |
| 10 uL pipette | Rainin | 17014388 | 1 | |
| 20 uL multichannel | Rainin | 17013803 | 1 | |
| pipette | | | | |
| 200 uL pipette tips | Corning | 4138 | 1 | box |
| 20 uL pipette tips | Rainin | 30389274 | 1 | |
| 20 uL pipette | Rainin | 30389226 | 1 | |
| tip box | | | | |
| Capillary PCR | Custom made | N/A | 1 | |
| holder | | | | |
| QX200 Droplet | BioRad | 17005227 | 1 | |
| Generator | | | | |
| 1.5/2 mL tube | Chang | MiniR1D5ML | 1 | |
| holder | Bioscience | | | |
| PCR Tube Holder | Fisherbrand | 03-448-20 | 2 | |
| Gloves | Ansell | 6034301 | 2 | boxes |
| Waste basket | Bel-Art | F131930500 | 1 | |
| Kimwipes | Kimberly-Clark | 34155 | 1 | box |
| | Professional | | | |
| PCR Tracker Table | Custom made | N/A | 5/OR | Case |
| Timer | LinkDM | LinkDm- | 4 | |
| | | E6-NEW | | |
| Power strip | GE | 45192 | 1 | |
| Laptop Computer | Lenovo | 20X4S6JJ00 | 1 | |
| QX 200 ddPCR | BioRad | 1864003 | 1 | |
| Droplet Reader | | | | |
| Cart 3: Post-PCR | | | | |
| Table | | | | |
| Cart (24″ × 18″) | DuraSteel | SS-DR2418H | 1 | |
| Kimwipes | Kimberly-Clark | 34155 | 1 | box |
| | Professional | | | |
| Gloves | Ansell | 6034301 | 1 | box |
| 96-well ddPCR | BioRad | 12001925 | 1 box (25 | |
| plate stock | | | plates) | |
| Timer | LinkDM | LinkDm- | 1 | |
| | | E6-NEW | | |
| 200 uL pipette | Eppendorf | 3123000055 | 1 | |
| 200 uL pipette tips | Corning | 4138 | 1 | box |

| | | | |
|---|---|---|---|
| Waste basket | Bel-Art | F131930500 | 1 |
| Other Supplies | | | |
| Mini Refrigerator | Black + Decker | BCRK17B | 1 |

Although all the carts could fit in the operating room, in order to conserve space, the carts were placed in a side room near the operating room.

In the morning prior to each surgical case, the following were prepared:

1) UR-ddPCR reaction mix: In cases #1-3, this was prepared in two separate parts, A and B, that were stored at room temperature and combined immediately prior to each reaction in the operating room in an initial attempt to maximize reagent stability over the course of a prolonged surgery. This is referred to as intraoperative protocol version 1. Part A (7 μL/sample) contained the UR ddPCR IDH1 R132H assay primers and probes and part B (14 μL/sample) contained HaeIII, Aptamer HS Taq DNA Polymerase, and the ddPCR supermix for probes (no dUTP). Once parts A and B were combined, the composition of the UR-ddPCR reaction mix is identical to that used in UR-ddPCR laboratory experiments described above with 32x HS Taq concentration (0.8 U/μL) except with a final volume of 21 μL/reaction, since only 1 μL of DNA is added in the operating room assays. However, after obtaining lower positive droplet counts for UR-ddPCR in the operating room compared to UR-ddPCR in the laboratory, the method of preparing UR-ddPCR reaction mix for the remaining cases #4-22 was altered, which is referred to herein as, intraoperative protocol version 2. For those cases, the UR-ddPCR reaction mix was prepared in the same way and with the same composition as used in UR-ddPCR laboratory experiments described above with 25× HS Taq concentration (0.625 U/μL), except with a final volume of 21 μL/reaction. The HS Taq concentration was lowered to 25× instead of 32×, since the studies in the laboratory that high concentrations of polymerase can cause droplet instability. Notably, this change did not affect assay performance (FIG. 6E). 21 μL of the reaction mix was then pre-aliquoted into one well per strip tube, with a separate strip tube for each sample and control that will be run for the surgical case. During the surgical case, the pre-prepared strip tubes were stored at 4° C. in a mini refrigerator until use.

2. 22 μL of 1X ddPCR Buffer Control for Probes was added to all the remaining strip tube wells that would not be used for a tissue sample, i.e., the remaining 7 wells of each strip tube that do not contain the UR-ddPCR reaction mix.

3. Buffer ME was activated as described for laboratory UR-ddPCR.

4. To conserve space in the operating room, a TissueLyser LT instrument (Qiagen) was used for homogenization with the 12-tube adaptor (Qiagen), instead of the TissueLyser II instrument that we used in the laboratory. The instrument was set to a 50 Hz, 30 second program. 5. 200 μL of activated buffer ME and six 2.8 mm ceramic beads (Omni, cat #19-646-3) were added to each of a number of 2 mL DNA LoBind tubes (Eppendorf) corresponding to the anticipated number of tissue and control samples for that case. These tubes were stored at room temperature, and studies confirmed in a tissue-to-result UR-ddPCR experiment that activated buffer ME is stable at room temperature for at least 3 hours. Ceramic beads were used instead of steel beads for UR-ddPCR in the operating room, because these were found to homogenize tissue more effectively in the TissueLyser LT instrument.

6. A mini dry bath (Fisher, cat #14-955-218) was pre-heated with a 2 mL adaptor (Fisher, cat #14-955-225) to 98° C. for use in the UR-DNA extraction heat step.

7. Two 1-liter water baths were prapred, one at 95° C. and one at 62° C.

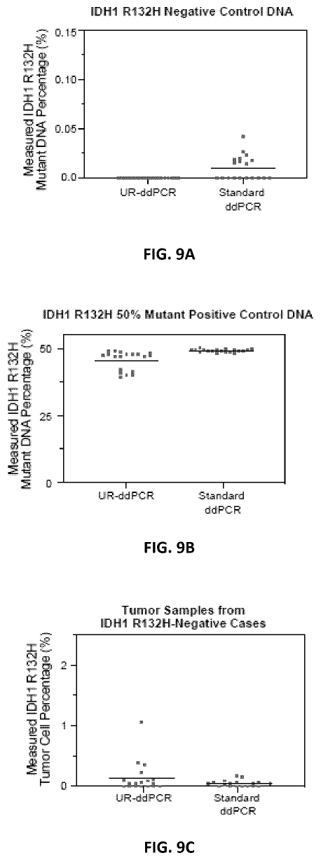

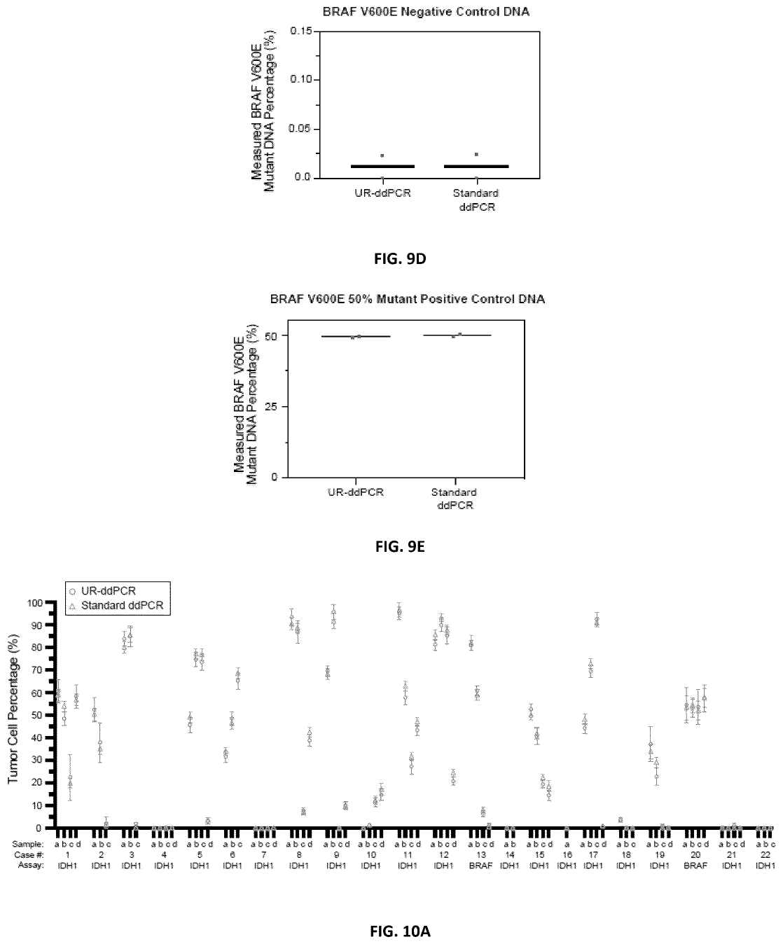

After these preparation steps were completed, two types of negative control assays were performed. The first negative control assay to exclude reagent contamination was performed by following the UR-DNA extraction process without any tissue input followed by UR-ddPCR. Across all cases, this yielded 0% average mutant DNA percentage for both UR-ddPCR and standard ddPCR (N=21, since 2 of 22 cases were conducted on the same day and shared a set of control experiments). Note, we assigned a 0% mutant DNA percentage to negative control samples with ≤3 total positive droplets (i.e., non-mutant plus mutant positive droplets), because this low number of positive droplets yields unreliable estimates of mutant DNA percentage. The second negative control assay performed measured the assay's false positive rate using 1 μL of 30 ng/μL non-mutant DNA (NA12877) added to the UR-ddPCR reaction mix. This yielded an average mutant DNA percentage for the IDH1 R132H cases of 0.00% and 0.01% (N=19) in UR-ddPCR and standard ddPCR, respectively, and an average mutant DNA percentage for the BRAF V600E cases of 0.01% and 0.01% (N=2) in UR-ddPCR and standard ddPCR, respectively (FIGS. 9A and 9D).

For cases 1-3 in which the UR-ddPCR mix was pre-prepared in two parts, approximately 5 to 10 minutes before each tissue sample was obtained in the operating room, 7 μL of the UR-ddPCR mix part A and 14 μL of UR-ddPCR mix part B were combined. For all cases, prior to each tissue sample becoming available, 70 μL of Droplet Generation Oil for Probes was also added to all DG8 cartridge oil wells, the QX Manager Software was initialized with the plate name and probes, and wells in the software were configured for analysis.

Upon resection of each approximately 8×8×2 mm tissue sample that was profiled in the operating room, the tissue was cut by the surgical team into two halves. One half was designated for profiling by the NIO Stimulated Raman Histology System¹⁵ (Invenio Imaging) followed by clinical pathology evaluation, and the other half was designated for UR-ddPCR. Additional tumor samples that were not profiled by ddPCR or NIO were also submitted for neuropathology evaluation.

Next, UR-DNA extraction was performed for each tissue sample by adding the tissue sample to the tube containing activated buffer ME and beads. UR-DNA extraction was then conducted per the UR-DNA extraction laboratory protocol except that homogenization used a TissueLyser LT instrument at 50 Hz for 30 seconds and the post-homogenization heat incubation was performed in the mini dry bath described above.

After UR-DNA extraction, 0.5 μL (case 1) or 1 μL (cases 2-22) of the UR-DNA extract was used as input into the UR-ddPCR reaction. The volume of UR-DNA extract was increased following case 1 to increase the numbers of positive droplets (mutant and non-mutant). The UR-ddPCR protocol was then followed as described above. For cases #1-3,capillaries from Component Supply Company were used and for the remaining cases, capillaries from Ziggy's Tubes and Wires were used, as detailed in the ‘Ultra-rapid ddPCR in the laboratory’ section. Additionally, during the droplet generation step, 20 μL of the UR-DNA extract of each sample was saved in a 1.5 mL DNA LoBind tube (Eppendorf) for later profiling by standard ddPCR. The above process was repeated for each tissue sample as it was resected.

Throughout the intraoperative UR-ddPCR process, the step-by-step times were measured using timers present at each station. The total time was calculated as the time between beginning the bead homogenization to the time when results were available after the sample was read on the droplet reader. The time of walking from the operating room to the side room was also measured, but this was excluded from the reported “total tissue-to-result time” since it is dependent on the specific operating room layout, and this time could be eliminated by placing all the instruments in the operating room.

Once the surgical day was completed, a positive control assay was performed by adding 1 μL of 30 ng/μL reference genomic DNA containing a 50% mutant DNA percentage (either IDH1 R132H or BRAF V600E depending on the case's target assay) to the UR-ddPCR reaction mix instead of UR-DNA extract and performing the UR-ddPCR intraoperative protocol beginning at droplet generation. This yielded an average mutant DNA percentage for the IDH1 R132H cases of 46% and 49% (N=19) in UR-ddPCR and standard ddPCR, respectively, and an average mutant DNA percentage for the BRAF V600E cases of 49% and 50% (N=2) in UR-ddPCR and standard ddPCR, respectively (FIGS. 9B and 9E).

Finally, standard ddPCR was conducted in the laboratory for all of the controls and tissue samples of the case. This standard ddPCR was conducted as previously described using 1 μL input of either control DNA or UR-DNA extract.

Additional samples from each case underwent neuropathology profiling by: 1) hematoxylin and eosin slides, 2) immunohistochemistry for IDH1 R132H (Dianova, GDIA-H09), 3) clinical test that performs whole-genome DNA methylation profiling and classification with the Heidelberg Classifier v1238, and 4) mutation and copy number analysis using the FDA-cleared NYU Langone Genome PACT matched tumor-normal DNA next-generation sequencing assay (FDA 510 (k): K202304).

### Quantification and Statistical Analysis

The ddPCR results were analyzed with the QX Manager 2.1 Standard Edition Software (Bio-Rad). QX Manager 2.1 analysis was conducted using the “Advanced Classification Method”. This analysis method allows for unclassified droplets that are excluded from calculations. Double negative (MUT−/WT−) droplets were first selected based on the heat map 2D amplitude plot with a boundary surrounding the main population density contour up until the region where individual droplets can be distinguished. The mutant positive (MUT+/WT−) and wildtype positive (WT+/MUT−) droplet populations were then marked. When mutant positive and wildtype positive populations lacked clear separation from the double negative population, these were marked as regions beginning 500 units away from and parallel to the double negative population edge along their respective axes on the 2D amplitude plot. Double positive droplets and ambiguous droplets (i.e., within 500 units from the double negative population) were left unclassified. Excluding double positive droplets from the analysis increases assay specificity at the cost of sensitivity loss, since double positive droplets are enriched for false-positives due to DNA damage^{39,40}. The mutant DNA percentages were then obtained from the QX Manager 2.1 software's data table in the “fractional abundance” column with Poisson 95% confidence intervals obtained from the “PoissonFractionalAbundanceMin” and “PoissonFractionalAbundanceMax” columns. For all intraoperative samples, tumor cell percentage was estimated as 2 × mutant DNA percentage. The Poisson 95% confidence intervals for tumor cell percentage were then calculated as 2 × PoissonFractionalAbundanceMin to 2 X PossionFractionAbundanceMax. These tumor cell percentage estimates assume that, in all tumor cells, the assayed mutation is heterozygous and that the mutation locus has a copy number of 2, which is true for the large majority of IDH1 mutant tumors^{5,41} and for the majority of BRAF V600E mutant tumors⁴².

Due to large error in estimating mutant DNA percentages when there are low positive droplet counts, samples with <100 total positive droplets (e.g., IDH1 R132H-mutant plus IDH1 non-mutant positive droplets) were excluded from analysis and plots. This low positive droplet count can occur due to variability in tissue sample size and cellularity, and it occurred for only 2 of 80 intraoperative tissue samples in this study.

Mutation status was confirmed by subsequent clinical sequencing for all cases, and absence of copy number changes was confirmed by methylation profiling for all but two cases (cases 19 and 21) that had non-informative methylation profiling. Future UR-ddPCR assays could feasibly also measure copy number of target loci⁴³.

### Results

### Ultra-Rapid DNA Extraction

Standard DNA extraction from tissues typically requires more than 30 minutes to perform. Therefore, the first step to develop an Ultra-Rapid (UR) ddPCR assay feasible for intraoperative use was to create an UR-DNA extraction method that is compatible with ddPCR. This is challenging because nearly all DNA extraction lysis buffers contain detergents that interfere with ddPCR droplet formation and stability. Two commonly used rapid DNA extraction buffers were initially tested-Buffer DL from the SwiftX DNA extraction kit and Lucigen QuickExtract solution-by adding them to a ddPCR reaction mix prior to droplet generation. However, neither of these buffers was compatible with ddPCR droplet formation (FIG. 1B). In contrast, a detergent-free DNA extraction buffer, SwiftX Buffer ME, maintained ddPCR droplet integrity (FIG. 1B). Using SwiftX Buffer ME, a 3-step UR-DNA extraction protocol was then developed-bead homogenization for 30 seconds in the presence of SwiftX Buffer ME, heat incubation at 98° C. for 2.5 minutes, and a brief 10-second centrifugation to separate cellular debris from clarified lysate containing DNA-that achieved the goal of 5 minutes for processing one tissue sample (FIGS. 1C, D and Methods).

The compatibility of the UR-DNA extraction protocol with ddPCR was confirmed by performing a standard ddPCR assay with input from either a standard DNA extraction or an UR-DNA extraction. UR-DNA extraction slightly reduced the ddPCR signal level and the separation between positive and negative droplet populations, but it did not affect the total number of ddPCR droplets (FIGS. 1E,F). Additionally, the UR-DNA extraction method can be scaled to process four samples in parallel in 7 minutes (FIG. 1D). Therefore, the UR-DNA extraction achieves the first step for UR-ddPCR assay when applied to tissues.

Ultra-Rapid ddPCR Thermal Cycling

To enable intraoperative use, the total time of a tissue-to-result UR-ddPCR assay should be less than 20 minutes. However, standard ddPCR thermal cycling, the longest step of ddPCR, takes ˜2 hours. Therefore, UR-ddPCR required a drastic reduction in ddPCR thermal cycling time to less than 5 minutes, which combined with UR-DNA Extraction (˜5 minutes), droplet generation (˜3 minutes), and droplet reading (˜3 minutes), would total less than 20 minutes. Using IDH1 R132H as the target assay (Table 1) and DNA extracted from an IDH1 R132H-mutant tumor, every aspect of ddPCR thermal cycling was changed, to achieve this goal.

The ddPCR thermal cycling time was reduced by shortening or removing each step of the standard ddPCR protocol and observing the ddPCR signal level after each successive change. Removing the 10-minute enzyme inactivation step, reducing the denaturation time from 30 seconds to 1 second, and reducing the annealing/extension step from 60 seconds to 15 seconds either maintained or only slightly reduced the ddPCR signal level and the separation between droplet populations (FIG. 2A). However, removing the 10-minute heat activation step led to complete loss of the ddPCR signal (FIG. 2A). To rescue the ddPCR signal in the absence of a heat activation step, an aptamer-inhibited hot-start Taq polymerase (Aptamer HS Taq) that is immediately activated above 45° C. was added to the reaction, in contrast to the prolonged heat activation step required by the standard ddPCR polymerase. This allowed elimination of the heat activation step while retaining the ability to generate signal (FIG. 2A).

Collectively, the above changes reduced the total thermal cycling time from 107 minutes to 37 minutes, but 25 minutes of this remaining time was due to the time spent by the thermal cycler instrument ramping between temperatures. Since even thermal cycler instruments with the most rapid ramping speeds would not allow for the desired total tissue-to-result ddPCR time, the protocol was modified by switching to a stainless-steel capillary water bath thermal cycling system (FIG. 2B). The stainless-steel capillary both increases the surface area to volume ratio of the reaction and increases the thermal conductance relative to a standard ddPCR reaction utilizing polypropylene reaction plates (FIG. 2B and Methods). This stainless-steel capillary system reduced the total ramping time to 1.5 minutes and was limited only by the speed of the technician moving the capillary between the water baths. Combined with the prior protocol changes, this reduced the total thermal cycling time to 12 minutes while maintaining significant ddPCR signal level and droplet population separation (FIG. 2C).

The last challenge in decreasing the thermal cycling time was to further reduce the annealing/extension step to less than 15 seconds. Initial steps attempted to shorten this step while increasing from 40 to 45 PCR cycles, however, ddPCR droplet population separation with annealing/extension times below 7 seconds was not attainable (FIG. 6A). Extending Extreme PCR18 principles to the ddPCR reaction resulted in a reduction in the annealing/extension step to only 1 second by (a) increasing the number of PCR cycles to 45, (b) increasing the Aptamer HS Taq concentration 32-fold, and (c) increasing the concentrations of primers and probes 4-fold (FIG. 2D and FIGS. 6B-D).

The careful selection of various parameters as discussed herein yielded an unprecedented total ddPCR thermal cycling time of 3 minutes (FIG. 2E), which meets the time constraints necessary for a UR-ddPCR assay.

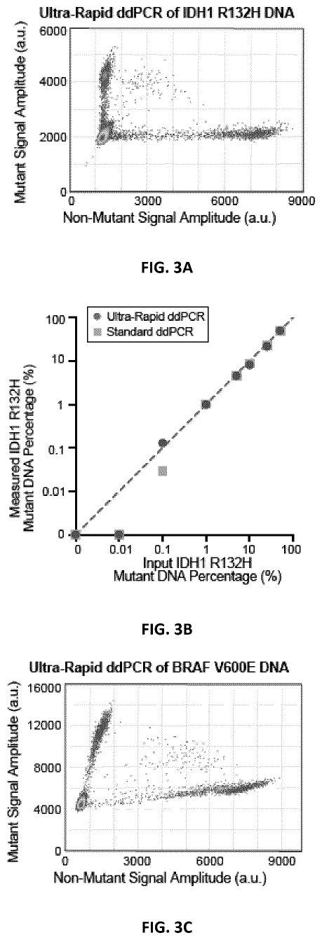

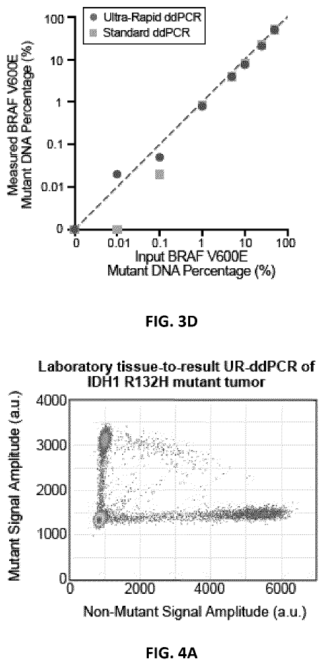

To confirm that UR-ddPCR maintained the high sensitivity and specificity of standard ddPCR, DNA standards with known IDH1 R132H mutant DNA percentages between 50% and 0.01% were assayed using both standard and UR-ddPCR. The results showed that both standard and UR-ddPCR assay accurately measured the mutant DNA percentage down to 0.1% and measured a mutant DNA percentage of 0% for non-mutant control DNA (FIGS. 3A, B and FIG. 6E).). In this experiment, the percentage of droplets that were positive for either mutant or non-mutant DNA was lower in UR-ddPCR (15%) than in standard ddPCR (28%). This suggests that UR-ddPCR does not amplify all the droplets containing target DNA, but that mutant and non-mutant droplets amplify proportionally such that the mutant DNA percentage is accurate and concordant with standard ddPCR. Altogether, these results demonstrate that UR-ddPCR provides ultra-rapid speed without sacrificing the high sensitivity and specificity of standard ddPCR.

In addition to the IDH1 R132H mutation, an UR-ddPCR assay for the BRAF V600E mutation was developed (Table 1). After careful selection of multiple assay parameters as disclosed herein, this assay achieved similar signal separation as the IDH1 R132H UR-ddPCR assay with similar conditions except it uses fewer PCR cycles (40) and longer annealing/extension time (5 seconds) for a total thermal cycling time of 6 minutes (FIG. 3C). The BRAF V600E UR-ddPCR assay also matched the sensitivity and specificity of standard ddPCR by measuring mutant DNA percentage down to 0.1% and measuring a mutant DNA percentage of 0% for non-mutant control DNA (FIG. 3D). These results demonstrate that UR-ddPCR is generalizable to other hotspot mutations. Tissue-to-Result Ultra-Rapid ddPCR in the Laboratory

Next, the UR-DNA extraction and UR-ddPCR was combined in a streamlined tissue-to-result UR-ddPCR. This process was first tested in the laboratory on 15 tumor samples obtained from two IDH1 R132H-mutant oligodendrogliomas. These 15 samples were profiled in six experiments: three experiments profiling one sample at a time, and three experiments each profiling four samples in parallel.

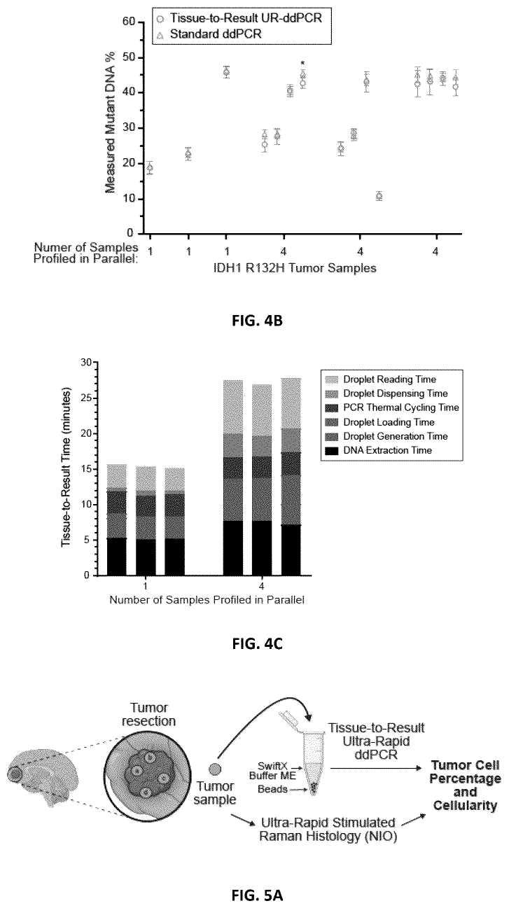

In every experiment profiling these samples, tissue-to-result UR-ddPCR achieved ddPCR signal levels similar to the prior UR-ddPCR profiling of purified DNA (FIGS. 3A and 4A). Additionally, mutant DNA percentages measured by tissue-to-result UR-ddPCR and by standard ddPCR of the same UR-DNA extraction lysates were highly concordant (FIG. 4B). Only 1 of the 15 samples showed a statistically significant difference between UR-ddPCR and standard ddPCR mutant DNA percentage measurements, and the absolute difference in this measurement was only 2.9% (FIG. 4B and Table 2). These tissue-to-result UR-ddPCR results were achieved in an average of 15 minutes and 20 seconds when profiling one sample at a time (N=3 experiments) and an average of 27 minutes and 25 seconds when testing 4 samples in parallel (N=3 experiments) (FIG. 4C). These results indicate that tissue-to-result UR-ddPCR can identify a tumor genetic subtype and quantify mutant DNA percentage with high accuracy and ultra-rapid speed.

Intraoperative Ultra-Rapid ddPCR

Since the ultimate goal in developing tissue-to-result UR-ddPCR was its use as a real-time guide for surgeons during operations, it was implemented in the operating room in 22 adult brain tumor cases. An efficient layout of all the items required for UR-ddPCR on mobile carts was first designed (FIG. 8A-8C, Table 3). To fully assess UR-ddPCR performance, every sample was profiled with both UR-ddPCR and then with standard ddPCR after the operation was over. Additionally, on each day in the operating room, the assay's performance was confirmed with two negative control assays, one without DNA input and one with non-mutant DNA input, and with a positive control assay of control DNA with a known (50%) mutant DNA percentage (Methods). The non-mutant DNA negative control UR-ddPCR assays measured an average 0.00% and 0.01% mutant DNA percentage in the IDH1 R132H and BRAF V600E assays, respectively (FIGS. 9A and 9D). The 50% mutant DNA positive control UR-ddPCR assays measured an average 46% and 49% mutant DNA percentage in the IDH1 R132H and BRAF V600E assays, respectively (FIGS. 9B and 9E).

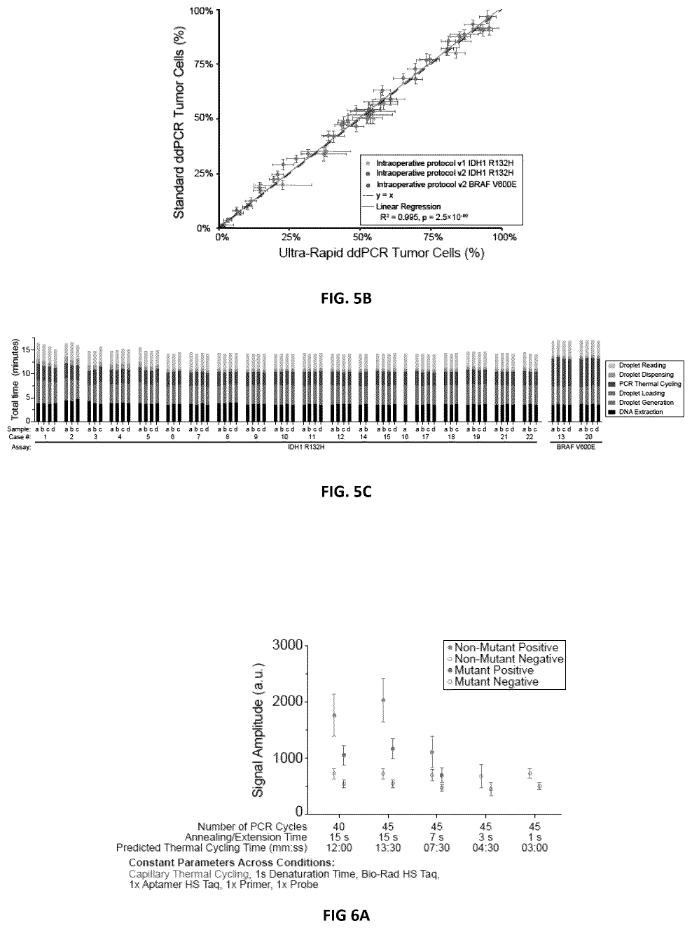

Each tissue sample provided by the surgeon for intraoperative diagnostics (average specimen size ˜8×8×2 mm) in the operating room was first split into approximately two halves: one half for UR-Stimulated Raman Histology15 (NIO system), which images the tissue in 5 minutes, followed by neuropathology analysis, and the other half for parallel profiling by UR-ddPCR (FIG. 5A).

Multiple tissue samples were assayed per case, including core and tumor margin samples, for a total of 78 samples across the 22 surgical cases. Tumor cell percentages measured by intraoperative UR-ddPCR were highly concordant with standard ddPCR performed on the same sample lysates (FIG. 5B). These UR-ddPCR results were achieved in an average of 14 minutes and 39 seconds for IDH1 R132H assays (N=70 samples, each processed individually) and an average of 17 minutes and 7 seconds for the BRAF V600E assays (N=8 samples, each processed individually) (FIG. 5C). These results demonstrate that intraoperative UR-ddPCR can both identify tumor genetic subtype and quantify tumor cell percentage with high accuracy and ultra-rapid speed.

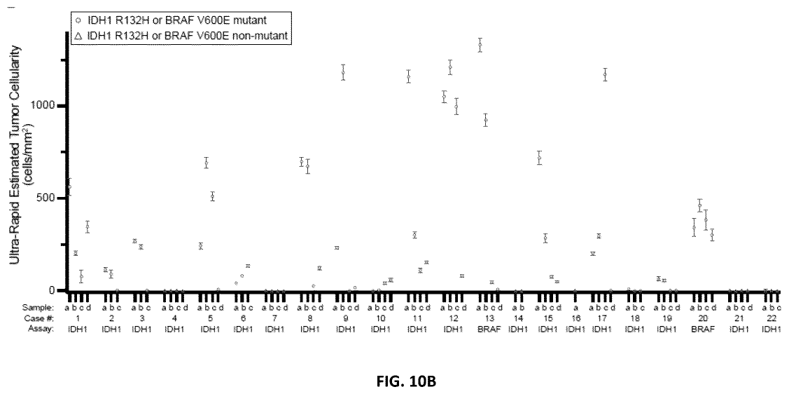

Since stimulated Raman histologic images acquired intraoperatively in minutes provided real-time measurements of total cellularity (cells/mm²), they also provided the ability to multiply these measurements with the UR-ddPCR measurements of tumor cell percentage to obtain ultra-rapid estimates of mutant tumor cellularity (i.e., tumor cells/mm²). Notably, individual cases had a wide range of tumor cell percentage and tumor cellularity estimates, consistent with the wide dynamic range of the assay (FIGS. 10A and 10B, FIGS. 3B and 3D, Table 4).

TABLE 4
Intraoperative cases

| | | | Sample Information | | | |
|---|---|---|---|---|---|---|
| | | | | | | Tissue- |
| | | | | Std | UR | to-Result |
| Case Information | | | | ddPCR | ddPCR | UR ddPCR |
| Case | Patient | ddPCR Assay | | Tumor | Tumor | Time |
| Number | Sex | Target | Sample | Cell % | Cell % | (min:s) |
| 1 | F | IDH1 R132H | a | 59.0 | 60.6 | 16:41 |
| | | | b | 54.0 | 48.4 | 15:55 |
| | | | c | 19.9 | 22.6 | 15:48 |
| | | | d | 56.8 | 58.4 | 15:14 |
| 2 | F | IDH1 R132H | a | 50.4 | 52.4 | 16:29 |
| | | | b | 35.2 | 38.0 | 15:44 |
| | | | c | 1.66 | 2.08 | 16:18 |
| 3 | M | IDH1 R132H | a | 80.0 | 83.8 | 14:57 |
| | | | b | 85.4 | 84.8 | 14:55 |
| | | | c | 0.22 | 1.99 | 15:51 |
| 4 | M | IDH1 R132H | a | 0.05 | 0.12 | 15:01 |
| | | | b | 0.00 | 0.05 | 15:04 |
| | | | c | 0.05 | 0.35 | 15:32 |
| | | | d | 0.16 | 0.00 | 15:13 |
| 5 | F | IDH1 R132H | a | 49.2 | 45.6 | 14:41 |
| | | | b | 77.2 | 74.6 | 14:39 |
| | | | c | 76.8 | 73.4 | 14:33 |
| | | | d | 3.74 | 3.14 | 14:39 |
| 6 | M | IDH1 R132H | a | 34.0 | 31.6 | 14:23 |
| | | | b | 46.6 | 48.6 | 14:25 |
| | | | c | 68.6 | 65.2 | 14:27 |
| 7 | F | IDH1 R132H | a | 0.00 | 0.05 | 14:38 |
| | | | b | 0.00 | 0.05 | 14:24 |
| | | | c | 0.06 | 0.06 | 14:33 |
| | | | d | 0.18 | 0.00 | 14:23 |
| 8 | M | IDH1 R132H | a | 90.4 | 93.4 | 14:32 |
| | | | b | 88.6 | 86.8 | 14:24 |
| | | | c | 7.02 | 7.50 | 14:33 |
| | | | d | 42.4 | 38.8 | 14:38 |
| 9 | M | IDH1 R132H | a | 68.2 | 69.6 | 14:34 |
| | | | b | 91.6 | 95.6 | 14:21 |
| | | | c | 0.05 | 0.07 | 14:23 |
| | | | d | 9.88 | 10.1 | 14:19 |
| 10 | M | IDH1 R132H | a | 0.00 | 0.00 | 14:19 |
| | | | b | 1.53 | 1.30 | 14:19 |
| | | | c | 12.4 | 11.4 | 14:24 |
| | | | d | 17.3 | 14.7 | 14:20 |
| 11 | M | IDH1 R132H | a | 96.6 | 95.0 | 14:31 |
| | | | b | 63.0 | 57.8 | 14:23 |
| | | | c | 31.8 | 27.4 | 14:25 |
| | | | d | 47.2 | 43.4 | 14:35 |
| 12 | F | IDH1 R132H | a | 85.6 | 81.2 | 14:21 |
| | | | b | 93.2 | 89.8 | 14:20 |
| | | | c | 87.6 | 85.2 | 14:17 |
| | | | d | 24.6 | 20.8 | 14:18 |
| 13 | F | BRAF V600E | a | 82.0 | 80.8 | 17:01 |
| | | | b | 59.0 | 60.8 | 17:13 |
| | | | c | 8.12 | 6.34 | 17:07 |
| | | | d | 0.72 | 1.41 | 17:04 |
| 14 | M | IDH1 R132H | a | 0.05 | 0.00 | 14:19 |
| | | | b | 0.00 | 0.00 | 14:22 |
| 15 | M | IDH1 R132H | a | 50.0 | 52.6 | 14:24 |
| | | | b | 42.0 | 40.6 | 14:23 |
| | | | c | 22.4 | 19.4 | 14:20 |
| | | | d | 18.5 | 14.5 | 14:29 |
| 16 | M | IDH1 R132H | a | 0.06 | 0.10 | 14:24 |
| 17 | F | IDH1 R132H | a | 48.2 | 44.2 | 14:21 |
| | | | b | 72.8 | 69.4 | 14:19 |
| | | | c | 91.0 | 92.4 | 14:14 |
| | | | d | 1.17 | 0.65 | 14:15 |
| 18 | M | IDH1 R132H | a | 4.18 | 3.70 | 14:31 |
| | | | b | 0.09 | 0.24 | 14:18 |
| | | | c | 0.05 | 0.17 | 14:29 |
| 19 | F | IDH1 R132H | a | 34.0 | 37.2 | 14:28 |
| | | | b | 29.2 | 22.8 | 14:24 |
| | | | c | 0.16 | 0.90 | 14:21 |
| | | | d | 0.12 | 0.38 | 14:24 |
| 20 | F | BRAF V600E | a | 53.4 | 54.6 | 17:10 |
| | | | b | 54.4 | 53.0 | 17:08 |
| | | | c | 52.0 | 53.6 | 17:10 |
| | | | d | 57.8 | 57.4 | 17:06 |
| 21 | M | IDH1 R132H | a | 0.00 | 0.38 | 14:22 |
| | | | b | 0.06 | 0.00 | 14:27 |
| | | | c | 0.09 | 1.06 | 14:28 |
| | | | d | 0.05 | 0.22 | 14:27 |
| 22 | M | IDH1 R132H | a | 0.00 | 0.00 | 14:36 |
| | | | b | 0.00 | 0.09 | 14:25 |
| | | | c | 0.03 | 0.00 | 14:21 |

For example, in case 5, tumor cell percentages of 45.6%, and 74.6% were measured for two core tumor samples and 73.4%, and 3.1% for two margin samples, respectively, which, combined with UR-histology cellularity measurements, estimated 243, 692, 511, and 7 tumor cells/mm², respectively. Subsequent clinical sequencing of all cases was concordant with the UR-ddPCR results (Table 4). The cases profiled included four IDH non-mutant tumors, one IDH2 mutant tumor, and one IDH1 R132C mutant tumor, whose tissues had an average 0.14% and 0.05% tumor cell percentage measured by UR-ddPCR and standard ddPCR, respectively, with one outlier UR-ddPCR tissue sample with 1% tumor cell percentage, indicating that measurement of >0.5% tumor cell percentage in >1 sample is needed to reliably call a case as IDH1 R132H mutant (FIG. 9C, FIGS. 10A and 10B). Overall, the workflow disclosed herein demonstrates an unprecedented ability to rapidly map tumor cell content during surgery.

### Discussion

Surgeons synthesize anatomic, physiologic, radiographic, and histologic data to create an operative strategy during tumor resection. To date, however, there has been no intraoperative method to rapidly and iteratively utilize tumor-specific genetic alterations to guide surgical resections. While ultrasound, intraoperative MRI, fluorescent markers¹⁹⁻²¹, and frozen histology help guide some surgeries, they are too slow for iterative use by surgeons, and they do not target definitive molecular markers. To capitalize on clonal hotspot mutations that define many human malignancies²², a strategy is needed to streamline intraoperative diagnosis and thus enhance the precision of tumor resection through ultra-rapid ddPCR. The studies herein show the development and validation of an ultra-rapid ddPCR technology that achieves these goals, with detection down to 0.1% tumor cell percentage and <1 tumor cell per mm² of tissue in 15 minutes for a single sample, and in under 30 minutes for 4 samples profiled in parallel. Based on background signal seen in IDH1 R132H-negative cases assayed, in practice, reliable calling of cases as IDH1 R132H-mutant requires measurement of >0.5% tumor cell percentage in more than one sample. Importantly, the use of this technology in the operating room was validated, demonstrating the fastest-reported intraoperative quantification of tumor cells.

Existing molecular methods proposed for intraoperative use include methylation profiling²³, quantitative PCR16, targeted DNA sequencing¹⁷, and Crispr-Cas12a assays²⁴.

While intraoperative methylation profiling can distinguish a wide range of tumor subtypes, it takes 40 minutes to perform, it cannot classify samples with low tumor purity, and the technology has limited range and accuracy in quantifying tumor cell percentage²³. Quantitative PCR, which has been demonstrated intraoperatively in ˜60 minutes from tissue to result¹⁶, also has limited accuracy compared to ddPCR25. Targeted DNA sequencing can quantify tumor cell percentage in a single tumor sample in ˜30 minutes¹⁷, however, this time-to-result is not rapid enough for repeated use in the same surgical case. Crispr-Cas12a assays take ˜60 minutes and provide only a qualitative measurement of tumor cell burden²⁴

In contrast to these methods, the disclosed tissue-to-result UR-ddPCR provides both quantitative measurement of tumor cell burdens and a superior speed sufficient for repeated use in the operating room.

The demonstration of UR-ddPCR assays for both IDH1 R132H and BRAF V600E hotspot mutations shows this technique can be extended to a broader array of clinically relevant genetic loci. Lim.

UR-ddPCR is well-positioned to complement the growing array of surgical adjuncts developed to assess completeness of resection, including navigation, ultrasound, intraoperative magnetic resonance imaging, fluorescent markers¹⁹⁻²¹, and Raman-and AI-based methods^{15,30}. With a growing understanding of the correlation between the completeness of tumor resection and clinical outcome^{10-12,31}, methods that can directly quantify tumor cell infiltration are critically important so that resection boundaries are safely maximized. Moreover, for brain tumors, as it becomes clear that the impact of surgical resection varies based on glioma molecular subtype³, there is a need for rapid and accurate molecular diagnostics at the time of surgery. UR-ddPCR meets this need.

Additionally, molecular diagnosis and/or quantification of tumor infiltration at the margins during the primary tumor resection may in the future inform stratification of patients to different targeted and chemotherapy treatments. For example, ascertaining BRAF mutation status hold great value in adults who present with suspected craniopharyngioma where BRAF V600E mutations are present in up to 90% of tumors32. Given the high response rate of papillary craniopharyngiomas to BRAF-MEK inhibition32, surgical resection could be avoided in cases where BRAF V600E mutation is detected with UR ddPCR during an initial surgical sample.

Notably, UR-ddPCR can be extended in the future to other point-of care diagnostics such as infectious diseases. UR-ddPCR demonstrates the potential of emerging ultra-rapid molecular assays to create a new standard for point-of-care molecular diagnostics in medicine.

### REFERENCES

1. Samsom, et al. (2022). The Journal of Pathology 258, 179-188. https://doi.org/10.1002/path.5988.
2. Ossowski, et al. (2022). JCO Oncology Practice 18, e1874-e1884. 10.1200/op.22.00260.
3. Molinaro, et al. (2020). JAMA Oncology 6, 495-503. 10.1001/jamaoncol.2019.6143.
4. Huang, L. E. (2019). Friend or foe—IDH1 mutations in glioma 10 years on. Carcinogenesis 40, 1299-1307. 10.1093/carcin/bgz134.
5. Hartmann, et al. (2009). Acta Neuropathologica 118, 469-474. 10.1007/s00401-009-0561-9.
6. Di Nunno, et al. (2023). Frontiers in Oncology 12. 10.3389/fonc.2022.1067252.
7. Dankner, et al. (2018). Oncogene 37, 3183-3199. 10.1038/s41388-018-0171-x.
8. Gerstung, et al. (2020). Nature 578, 122-128. 10.1038/s41586-019-1907-7.
9. Suzuki, et al. (2015). Nature Genetics 47, 458-468. 10.1038/ng.3273.
10. Delev, et al. (2019). Journal of Neuro-Oncology 141, 223-233. 10.1007/s11060-018-03030-w.
11. Santamaria-Barria, et al. (2022). Current Oncology Reports 24, 1425-1432. 10.1007/s11912-022-01289-x.
12. Sanai, et al. (2018). Nature Reviews Clinical Oncology 15, 112-125. 10.1038/nrclinonc.2017.171.
13. Orringer, et al. (2012). Journal of Neurosurgery JNS 117, 851-859. https://doi.org/10.3171/2012.8.JNS12234.
14. Beiko, et al. (2013). Neuro Oncol 16, 81-91. 10.1093/neuonc/not159.
15. Hollon, et al. (2020). Nature Medicine 26, 52-58. 10.1038/s41591-019-0715-9.
16. Shankar, et al. (2015). JAMA Oncology 1, 662-667. 10.1001/jamaoncol.2015.0917.
17. Wadden, et al. (2022). Communications Biology 5, 708. 10.1038/s42003-022-03657-6.
18. Farrar, et al. (2015). Clinical chemistry 61, 145-153.
19. Hadjipanayis, et al. (2019). Journal of neuro-oncology 141, 479-486.
20. van Dam, et al. (2011). Nature Medicine 17, 1315-1319. 10.1038/nm.2472.
21. Tanyi, et al. (2022). Journal of Clinical Oncology 41, 276-284. 10.1200/JCO.22.00291.
22. Chang, et al. (2016). Nature Biotechnology 34, 155-163. 10.1038/nbt.3391.
23. Vermeulen, et al. (2023). Ultra-fast deep-learned CNS tumour classification during surgery. Nature 622, 842-849. 10.1038/s41586-023-06615-2.
24. Feng, et al. (2023). Scientific Reports 13, 5748. 10.1038/s41598-023-32957-y.
25. Wainman, et al. (2024). Applications of Digital Polymerase Chain Reaction (dPCR) in Molecular and Clinical Testing. The Journal of Applied Laboratory Medicine 9, 124-137. 10.1093/jalm/jfad103.
26. Youssef, et al. (2020). Lower Grade Gliomas. Current Neurology and Neuroscience Reports 20, 21. 10.1007/s11910-020-01040-8.
27. Satgunaseelan, et al. (2024). Pathology 56, 158-169. https://doi.org/10.10166.pathol.2023.11.003.
28. Makino, et al. (2021). BMC Cancer 21, 1025. 10.1186/s12885-021-08733-4.
29. Jeuken, et al. (2009). Brain Pathology 19, 661-671. https://doi.org/10.1111/1.1750-3639.2009.00320.x.
30. Nasrallah, et al. (2023). Med 4, 526-540.e524. 10.1016/j.medj.2023.06.002.
31. Massaad, et al. (2024). Neuro Oncol 26, 1660-1669. 10.1093/neuonc/noae073.
32. Brastianos et al. (2023). New England Journal of Medicine 389, 118-126. 10.1056/NEJMoa2213329.
33. Shen, et al. (2021). Lab on a Chip 21, 3742-3747.
34. Ren, et al. (2023). A three-in-one microfluidic droplet digital PCR platform for absolute quantitative analysis of DNA. Lab on a Chip. 10.1039/D3LC00107E.
35. Untergasser, et al. (2012). Primer3—new capabilities and interfaces. Nucleic Acids Research 40, e115-e115. 10.1093/nar/gks596.
36. Antonella, et al. (2019). Thermal Conductivity of Polypropylene-Based Materials. In Polypropylene, W. Weiyu, and Z. Yiming, eds. (IntechOpen), pp. Ch. 3. 10.5772/intechopen.84477.
37. Graves, et al. (1991). The thermal conductivity of AISI 304L stainless steel. International Journal of Thermophysics 12, 409-415. 10.1007/BF00500761.
38. Galbraith, et al. (2023). Clinical utility of whole-genome DNA methylation profiling as a primary molecular diagnostic assay for central nervous system tumors—A prospective study and guidelines for clinical testing. Neuro-Oncology Advances 5. 10.1093/noajnl/vdad076.
39. Milbury, et al. (2014). Determining lower limits of detection of digital PCR assays for cancer-related gene mutations. Biomolecular Detection and Quantification 1, 8-22. https: doi.org/10.1016/i.bdq.2014.08.001.
40. Henriksen, et al. (2022). Clinical Chemistry 68, 657-667. 10.1093/clinchem/hvab274.
41. The-Cancer-Genome-Atlas-Research-Network (2015). New England Journal of Medicine 372, 2481-2498. doi: 10.1056/NEJMoa1402121.
42. Hélias-Rodzewicz, et al. (2015). BMC Cancer 15, 497. 10.1186/s12885-015-1515-3.
43. Wolter, et al. (2022). Acta Neuropathologica Communications 10, 42. 10.1186/s40478-022-01335-6.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Those skilled in the art will recognize, or be able to ascertain, using no more than routine experimentation, many equivalents to the specific embodiments of the method and compositions described herein. Such equivalents are intended to be encompassed by the following claims.

We claim:

1. A method for ultra rapid droplet digital polymerase chain reaction (UR-ddPCR), comprising a process for ultra-rapid thermal cycling (UR-TC), the method comprising:
(i) contacting a sample comprising target nucleic acid sequences with an UR-ddPCR mix comprising a thermostable DNA polymerase, optionally a suitable restriction enzyme or a glycosylase enzyme, dNTPs, salts and buffer required for polymerase activity, primers configured for amplification of the target nucleic acid sequence, and either detection probes that are nucleic acids designed for detecting target nucleic acid sequences by fluorescence or a non-specific DNA quantification dye,
wherein (a) the polymerase is provided at a final reaction concentration between at least 0.15 U/μl to 1 U/μL (where 1U (unit) is the amount is the amount of enzyme that incorporates 15 nmol of dNTP into acid insoluble material in 30 minutes at 75° C.), (b) the primers are provided at a final reaction concentration between at least 1.8 μM to 10.8 μM each; and (c) the detection probes, if used, are provided at a final reaction concentration between at least 0.375 μM to about 3 μM each;
(ii) generating water-in-oil emulsion droplets comprising a water phase with the target nucleic acid sequences, polymerase, additional optional enzymes such as a restriction enzyme or a glycosylase enzyme, dNTPs, salts and buffer, primers, and either detection probes or a DNA quantification dye;
(iii) loading the droplets into a droplet container having a thermal conductance and surface area to volume ratio that is effective for UR-TC;
(iv) amplifying the target nucleic acid sequence by UR-TC by contacting the droplet container with heating elements through a plurality of PCR amplification cycles where each cycle is completed in about 2 to less than about 13 seconds, per cycle, with each PCR cycle comprising a denaturation step and either a combined annealing/extension step or separate annealing and extension steps, with the heating elements pre-heated prior to the start of PCR to the temperatures of the PCR steps,
wherein optionally, prior to UR-TC, the method comprises ultra-rapid nucleic acid extraction (UR-Nucleic acid (NA) extraction) comprising extracting nucleic acid from a sample by contacting the sample with a detergent-free nucleic acid extraction buffer.

2. The method of claim 1, wherein the sample is a tissue sample, and the UR-NA process comprises homogenizing the tissue sample in the presence of a detergent-free nucleic acid extraction buffer for about 30 seconds, incubating at a temperature of about 98° C. for about 2.5 minutes, and performing a brief about 10-second centrifugation at about 2,000×g to obtain an UR-NA sample.

3. The method of claim 1, wherein the nucleic acid is genomic DNA, optionally wherein the genomic DNA is eukaryotic DNA.

4. The method of claim 1, wherein the sample is selected from the group consisting of blood, urine, cerebrospinal fluid, seminal fluid, saliva, sputum, stool, and tissue.

5. The method of claim 2, wherein the tissue sample is a brain tissue sample.

6. The method of claim 1, wherein the polymerase is an aptamer-inhibited hot-start polymerase.

7. The method of claim 1, wherein the heating elements are selected from the group consisting of heated water (water baths), thermoelectric heating elements, resistive heating elements, radiating heating elements, heated oil and heated fluorocarbon-based liquids.

8. The method of claim 1 wherein the heating elements comprise a first water bath at a first temperature, optionally wherein the first temperature is about 85° C. to about 100° C., and a second water bath at a second temperature, optionally wherein the second temperature is about 50° C. to about 78° C.

9. The method of claim 1, wherein the droplet container has a thermal conductance of at least 1.5×105 W/(m²*K), and a surface area to volume ratio of at least 3.75 mm^{2/}μL.

10. The method of claim 1, wherein the droplet container is made from a material selected from the group consisting of steel, aluminum, copper, silver, glass, polypropylene, polycarbonate and silicon carbide.

11. The method of claim 9, wherein the droplet container is a stainless steel capillary tube having a thermal conductance of about 1.5×10⁵ W/(m²*K) and/or a surface area to volume ratio of about 3.75 mm²/μL.

12. The method of claim 1 wherein UR-TC comprises the following steps: (a) denaturation from about 1 second to about 3 seconds; and (b) a combined annealing/extension step from about 1 second to about 10 seconds.

13. The method of claim 1, wherein UR-TC does not include an enzyme activation step or an enzyme deactivation step.

14. A process performed in a surgical operating suite for detecting cancer hotspot mutations in a tissue sample obtained from a subject, comprising the following steps:
(a) ultra-rapid nucleic acid extraction (UR-Nucleic acid (NA) extraction) comprising extracting nucleic acid from a sample by contacting the sample with a detergent-free nucleic acid extraction buffer and homogenizing for about 30 seconds, incubating at a temperature of about 98° C. for about 2.5 minutes, and performing a brief about 10-second centrifugation at about 2,000×g to obtain an UR-NA sample; and
(b) ultra-rapid thermal cycling (UR-TC) comprising:
(i) contacting a sample comprising target nucleic acid sequences with an UR-ddPCR mix containing a thermostable DNA polymerase, optionally a suitable restriction enzyme or a glycosylase enzyme, dNTPs, salts and buffer required for polymerase activity, primers configured for amplification of the target nucleic acid sequence, and either detection probes designed for detecting target nucleic acid sequences by fluorescence or a non-specific DNA quantification dye,
wherein (a) the polymerase is provided at a final reaction concentration between at least 0.15 U/μl to 1 U/μL (where 1U (unit) is the amount is the amount of enzyme that incorporates 15 nmol of dNTP into acid insoluble material in 30 minutes at 75° C.), (b) the primers are provided at a final reaction concentration between at least 1.8 μM to 10.8 μM each; and (c) the detection probes, if used, are provided at a final reaction concentration between at least 0.375 μM to about 3 μM each;
(ii) generating water-in-oil emulsion droplets comprising a water phase with the target nucleic acid sequences, polymerase, additional optional enzymes such as a restriction enzyme or a glycosylase enzyme, dNTPs, salts and buffer, primers, and either detection probes or a DNA quantification dye;
(iii) loading droplets into a droplet container having a thermal conductance and surface area to volume ratio that is effective for UR-TC;
(iv) amplifying the target nucleic acid sequence by UR-TC comprising contacting the droplet container with heating elements through a plurality of PCR amplification cycles where each cycle is completed in about 2 to less than about 13 seconds, per cycle, with each PCR cycle comprising a denaturation step and either a combined annealing/extension step or separate annealing and extension steps, with the heating elements pre-heated prior to the start of PCR to the temperatures of the PCR steps.

15. The method of claim 14, wherein the mutation is selected from the group consisting of IDH1 R132H, IDH1 R132C, IDH1 R132S, IDH1 R132G and BRAF V600E.

16. The method claim 14, comprising homogenizing the tissue sample for about 30 seconds in the presence of a detergent-free nucleic acid extraction buffer.

17. The method of claim 15, wherein the mutation is selected from the group comprising IDH1 R132H, IDH1 R132C, IDH1 R132S, and IDH1 R132G, wherein the UR-NA is contacted with about 0.625 U/μL Hot Start (aptamer-based) Taq DNA Polymerase, about 3.6 μM each of a forward and reverse primer, and about 1 μM of each probe targeting the mutant and non-mutant nucleic acid sequences, wherein the method comprises performing about 45 cycles of 1 second denaturation in a 95° C. water bath and 1 second of annealing/extension in a 62° C. water bath.

18. The method of claim 15, wherein the mutation is BRAF V600E, wherein the UR-NA is contacted with about 3.6 μM of a forward and reverse primer and about 0.625 U/μL Hot Start (aptamer-based) Taq DNA Polymerase, wherein the method comprises performing about 40 cycles of 1 second denaturation in a 95° C. water bath and 5 seconds of annealing/extension in a 62° C. water bath.

19. The method of claim 15, further comprising contacting the UR-NA with a labelled probe designed to hybridize to the target nucleic acid sequence, wherein the probe includes locked nucleic acids.

20. The method of claim 14, wherein the tumor cell percentage measured by UR-ddPCR is multiplied by the total number of cells per mm² measured by histology to calculate an estimated number of tumor cells per mm².
